(19) United States
(12) Patent Application Publication
(10) Pub. No.: US 2021/0403897 A1
(43) Pub. Date: Dec. 30, 2021

(54) CHEMICAL ENGINEERING OF ERYTHROCYTES TO DISPLAY POLYPEPTIDES AND OTHER BIOMACROMOLECULES

(71) Applicant: Carnegie Mellon University, Pittsburgh, PA (US)

(72) Inventors: Weihang Ji, Pittsburgh, PA (US); Richard R. Koepsel, Pittsburgh, PA (US); Alan J. Russell, Pittsburgh, PA (US)

(21) Appl. No.: 17/290,032

(22) PCT Filed: Oct. 30, 2019
(86) PCT No.: PCT/US2019/058885

§ 371 Date: Apr. 29, 2021

Related U.S. Application Data
(60) Provisional application No. 62/766,690, filed on Oct. 30, 2018.

(51) Int. Cl. C12N 11/089; C12N 5/00; A61K 47/69; A61K 47/64; A61K 47/60

(52) U.S. Cl. CPC C12N 11/089; C12N 5/0006; A61K 47/60; A61K 47/64; A61K 47/6901

(57) ABSTRACT

Methods and materials for generating and using complexes that contain erythrocytes coupled to polypeptides, and particularly to erythrocyte-polypeptide complexes that can interact with biological molecules, are provided herein.

### CROSS-REFERENCE TO RELATED APPLICATIONS

This application claims benefit of priority from U.S. Provisional Application Ser. No. 62/766,690, filed Oct. 30, 2018, the contents of which are incorporated herein by reference in their entirety.

### TECHNICAL FIELD

This document relates to methods and materials for generating and using complexes that contain erythrocytes coupled to biomacromolecules (e.g., polypeptides), including erythrocyte-polypeptide complexes that can interact with biological molecules.

### BACKGROUND

Immunoglobulin-based drugs (IgG in particular) are remarkably successful therapeutics due to their exquisite specificity, strong antigen binding, circulatory biocompatibility, and acceptable serum half-lives (Nestorov, Semin Arth Rheum 34(5, Suppl 1):12-18, 2005). The most broadly used of these drugs are those that bind and sequester tumor necrosis factor alpha (TNFα) from circulating plasma. The biology of TNFα and the drugs that form what is known as a TNF blockade have been the subject of intense study (Sedger and McDermott, Cytokine Growth Factor Rev 25:453-72, 2014). The therapeutic benefit of antibody-TNFα binding in plasma can be compromised by the development of anti-drug antibodies, however, which may contribute to treatment failure in up to 30% of patients (Vincent et al., Ann Rheum Dis 72:165-178, 2013). Treatment failures with adalimumab (Humira), one of the most effective immunoglobulin drugs, can occur when plasma concentrations drop below 5 μg/mL in the presence of anti-drug antibodies (Laine et al., Biologics 10:67-73, 2016). The therapeutic efficacy of anti-TNF requires plasma concentrations of 8-10 μg/mL, meaning that this single species must represent 0.1% of the entire IgG population of 11 mg/mL (Gonzalez-Quintela et al., Clin Exp Immunol 151:42-50, 2008). Given the extremely high relative anti-TNF to IgG drug levels required for effectiveness, it is not surprising that nearly 30% of patients develop anti-anti-TNF antibodies (Laine et al., supra).

### SUMMARY

Erythrocytes (RBCs) can be used to deliver therapeutics. Most RBC-drug delivery constructs in clinical trials, however, have been made by encapsulation of the drugs. An osmotic swelling process opens pores in the RBC membrane, releasing hemoglobin and allowing the drug(s) of interest into the cells. The pores can be closed by return to isotonic conditions, leaving drug-filled, non-oxygen transferring erythrocyte ghosts (Villa et al., Transfusion Apheresis Sci 55:275-280, 2016). The osmotic shock can have serious effects on the stability of the RBC, however, altering deformability, reducing membrane stability, and increasing the sensitivity to a variety of stressors. These changes can reduce the biocompatibility of the cells and increase their rate of clearance (Villa et al., supra). Molecular biology-based science also has been used to express therapeutic proteins on the surface of RBCs by genetically modifying the hematopoietic stem cells (Han et al., Bioconjugate Chem 29:852-60, 2018). Such approaches also involve osmotic shock and its associated adverse effects.

This document is based, at least in part, on the development of a new, erythrocyte-based platform that can present immunoglobulin-based drugs (e.g., antibodies) in order to scavenge biological molecules (e.g., TNFα) from the circulation, or that can present enzymes in order to catalyze reactions on circulating biological molecules. In some embodiments, for example, this document provides erythrocyte constructs that include a polymer bound via a first moiety to an erythrocyte and via a second moiety to an antibody-binding polypeptide. Antibodies that bind to the construct via the antibody-binding polypeptide can retain their native antigen binding activities. Thus, in some embodiments, this document provides erythrocyte complexes in which a polymer is bound to a RBC and to an antibody-antigen complex via an antibody-binding polypeptide that interacts with the Fc region of an antibody, thus masking the antibody Fc region of the antibody. This can provide a very long lifespan in vivo, and can prevent antidrug-antibody interactions, as well as unpredictable Fc region interactions. As described herein, the erythrocyte complexes can remove physiologically relevant amounts of biological molecules, such as TNF-α, from the circulation. In some embodiments, this document also provides erythrocyte complexes in which a polymer is bound to a RBC and to a protein (e.g., an enzyme), where the engineered RBC can be used as a stable, long-lived delivery vehicle for protein replacement therapy. The erythrocyte constructs provided herein can interact with more than 10 million active antibodies or other biomacromolecules per cell, and can remain viable for at least 60 days under in vitro storage conditions. The constructs provided herein can be generated using a patient's own erythrocytes, and can circulate for much longer periods than current scavenging or enzyme replacement therapeutics.

In a first aspect, this document features a conjugate containing a RBC, a synthetic polymer having at least first and second coupling moieties, where the polymer is coupled to the RBC via the first moiety, and a biomacromolecule coupled to the polymer via the second moiety. The polymer can be coupled to an amino group on the RBC. The polymer can be, or can be derived from (i) N-hydrosuccinidyl ester-functionalized homobifunctional poly(ethylene glycol) (NHS-PEG-NHS), or (ii) azido-PEG-NHS ester (N3-PEG-NHS) and alkyne-PEG-NHS ester (Alk-PEG-NHS). The polymer can be coupled to a thiol group on the RBC. The polymer can be, or can be derived from (i) maleimide-PEG-NHS ester (MAL-PEG-NHS), or (ii) 2-iminothiolane hydrochloride (Traut's reagent) and MAL-PEG-NHS ester. The biomacromolecule can be a polypeptide (e.g., Staphylococcus aureus Protein A; SpA). The conjugate can further include a therapeutic polypeptide bound to the polymer-coupled biomacromolecule. The therapeutic polypeptide can be an antibody, the polymer-coupled biomacromolecule can bind to the antibody such that the Fc region of the antibody is masked. The conjugate can have polymer molecules coupled to the RBCs at a density of about 10⁴ to about 10⁸ polymer molecules per RBC.

In another aspect, this document features a method for coupling a biomacromolecule to a RBC, where the method includes (a) conjugating a polymer to the biomacromolecule via a first moiety of the polymer, where the polymer is MAL-PEG-NHS and wherein the first moiety is NHS, to generate a biomacromolecule-polymer-MAL conjugate, (b) coupling Traut's reagent to one or more amino groups on the RBC thus generating thiol groups on the RBC, and (c) coupling the biomacromolecule-polymer-MAL conjugate to a thiol group on the RBC via the MAL moiety of the polymer. Each of steps (a), (b), and (c) can be carried out in a separate reaction. The biomacromolecule can be a polypeptide (e.g., SpA). The method can further include contacting the polymer-conjugated biomacromolecule with a therapeutic polypeptide (e.g., an antibody). The polymer can be conjugated to the RBC at a density of about 10⁴ to about 10⁸ polymer molecules per RBC.

In another aspect, this document features a method for coupling a biomacromolecule to a RBC, where the method includes (a) conjugating a polymer to the biomacromolecule via a first NHS moiety of the polymer, where the polymer is NHS-PEG-NHS, and (b) coupling the polymer to an amino group on the RBC via a second NHS moiety of the polymer. Steps (a) and (b) can be carried out in a single, multistep reaction. The biomacromolecule can be a polypeptide (e.g., SpA). The method can further include contacting the polymer-conjugated biomacromolecule with a therapeutic polypeptide (e.g., an antibody). The polymer can be coupled to the RBC at a density of about 10⁴ to about 10⁸ polymer molecules per RBC.

In another aspect, this document features a method for coupling a biomacromolecule to a RBC, where the method includes (a) conjugating a polymer to the biomacromolecule via a first moiety of a polymer, where the polymer is MAL-PEG-NHS and where the first moiety is the NHS group, to generate a biomacromolecule-polymer-MAL conjugate, and (b) coupling the biomacromolecule-polymer-MAL conjugate to a thiol group on the RBC via the MAL moiety. Steps (a) and (b) can be carried out in separate reaction vessels. The biomacromolecule can be a polypeptide (e.g., SpA). The method can further include contacting the polymer-conjugated biomacromolecule with a therapeutic polypeptide (e.g., an antibody). The polymer can be coupled to the RBC at a density of about 10⁴ to about 10⁸ polymer molecules per RBC.

In yet another aspect, this document features a method for coupling a biomacromolecule to a RBC, where the method includes (a) conjugating a first click reagent to the biomacromolecule to generate a biomacromolecule-click reagent conjugate, where the first click reagent is azido-PEG-NHS ester (N3-PEG-NHS) or alkyne-PEG-NHS ester (Alk-PEG-NHS), (b) conjugating a second click reagent to the RBC to generate a RBC-click reagent conjugate, where the second click reagent is N3-PEG-NHS when the first click reagent is Alk-PEG-NHS, and where the second click reagent is Alk-PEG-NHS when the first click reagent is N3-PEG-NHS, and (c) coupling the polypeptide-click reagent conjugate to the RBC-click reagent conjugate, thus coupling the polypeptide to the RBC. Each of steps (a), (b), and (c) can be carried out in a separate reaction. The biomacromolecule can be a polypeptide (e.g., SpA). The method can further include contacting the polymer-conjugated biomacromolecule with a therapeutic polypeptide (e.g., an antibody). The polypeptide can be conjugated to the RBC at a density of about 10⁴ to about 10⁸ polypeptide molecules per RBC.

This document also features a method for modifying a RBC, where the method includes contacting the RBC with Traut's reagent such that one or more amino groups on the RBC are effectively converted to thiol groups.

In addition, this document features RBCs conjugated to Traut's reagent, and compositions containing RBCs coupled to Traut's reagent.

In another aspect, this document features a method for treating a mammal, where the method includes administering to the mammal a composition containing a RBC conjugate, where the RBC conjugate contains (i) a RBC, (ii) a polymer having at least first and second coupling moieties, where the polymer is coupled to the RBC via the first moiety, and (iii) a biomacromolecule coupled to the polymer via the second moiety. The biomacromolecule can be a polypeptide (e.g., SpA). The conjugate can further include an antibody bound to the SpA polypeptide. The antibody can be bound to the SpA polypeptide such that the Fc region of the antibody is masked. The method can further include, prior to the administering, removing RBCs from the mammal and generating the conjugate from the RBCs. In addition, the method can further include introducing the conjugate into the same mammal from which the RBCs were removed.

In another aspect, this document features a method for reducing adverse effects of antibody treatment, where the method includes attaching an antibody to SpA on the surface of a RBC. The SpA can be coupled to the RBC via a polymer.

This document also features a conjugate containing a RBC; a polymer having at least first and second coupling moieties, where the polymer is coupled to the RBC via the first moiety, and where the polymer is, or is derived from NHS-PEG-NHS, MAL-PEG-NHS, or Traut's reagent and MAL-PEG-NHS ester; SpA coupled to the polymer via the second moiety; and an anti-TNF antibody associated with the SpA.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic illustrating RBC-PEG-SpA-Ab complexes removing antigens in vivo. Therapeutic IgG antibodies are displayed on the surface of RBCs through membrane engineering via a PEG linker and Protein A, and are bound via the Fc portion. The RBC-PEG-SpA-antibody complexes can circulate in vivo and scavenge target antigen molecules.

FIGS. 2A-2D are schemes depicting four synthetic routes of generating RBC-PEG-SpA constructs, using three different conjugation approaches. FIG. 2A depicts a reaction scheme using direct amine coupling with NHS-PEG-NHS. FIG. 2B depicts a reaction scheme using direct thiol coupling with NHS-PEG-MAL. FIG. 2C depicts a reaction scheme using coupling to converted thiols with NHS-PEG-MAL. FIG. 2D depicts a reaction scheme using click chemistry for direct amine coupling with NHS-PEG-Azido and RBC-PEG-alkyne.

FIG. 3 is a graph plotting the reaction progress of hydrolysis and aminolysis of NHS-mPEG

FIG. 4 shows MALDI-TOF spectra of SpA (native) and SpA-Br conjugates, as indicated.

FIGS. 5A and 5B indicate reaction rates for SpA with NHS-PEG-NHS. FIG. 5A is an image showing SDS-PAGE of the reaction between NHS-PEG-NHS and SpA. 1.0 mg of SpA and 1.0 mg of PEG were mixed, and timed samples containing 5 μg SpA were removed, diluted into Tris-glycine-SDS loading buffer, and loaded onto a 7.5% PAGE gel. FIG. 5B is a graph plotting band densities calculated relative to the unreacted SpA, as determined by scanning the gel.

FIG. 6 is an image of an SDS-PAGE gel with reaction products between SpA and NHS-PEG₅₀₀₀-MAL at different time intervals. Lane 1, ladder; Lane 2, SpA; Lane 3, [PEG]: [SpA]=8:1, 10 minutes; Lane 4, [PEG]:[SpA]=8:1, 30 minutes; Lane 5, [PEG]:[SpA]=8:1, 60 minutes; Lane 6, [PEG]:[SpA]=16:1, 10 minutes; Lane 7, [PEG]: [SpA]=16:1, 30 minutes; Lane 8, [PEG]: [SpA]=16:1, 60 minutes; Lane 9, ladder.

FIG. 7 is an image of an SDS-PAGE gel of reaction products between SpA and NHS-PEG₂₀₀₀-azido at different time intervals. Lane 1, ladder; Lane 2, SpA; Lane 3, SpA with glycine; Lane 4, [PEG]:[SpA]=8:1, 10 minutes; Lane 5, [PEG]:[SpA]=8:1, 30 minutes; Lane 6, [PEG]:[SpA]=8:1, 60 minutes; Lane 7, [PEG]:[SpA]=16:1, 10 minutes; Lane 8, [PEG]:[SpA]=16:1, 30 minutes; Lane 9, [PEG]:[SpA]=16:1, 60 minutes; Lane 10, ladder.

FIGS. 8A and 8B show the functionality of SpA-RBC antibody conjugates. Binding between an anti-TNF-α antibody and the specific antigen (TNF-α) was observed using confocal imaging of fluorescently labeled antibody and antigen. The SpA-RBC antibody conjugates tagged with ALEXA FLUOR® 488 were incubated with TNF-α tagged with ALEXA FLUOR® 647 for 2 hours with rotating. Samples were spun down and washed prior to imaging. FIG. 8A includes representative images of unmodified hRBCs. FIG. 8B includes representative images of RBC conjugates unbound and bound with scavenged TNF-α. After washing, fluorescent images were captured on a Zeiss LSM 880 Meta FCS confocal microscope at 63×. Brightness was adjusted to 40% for display purposes. Scale bars, 20 μm.

FIGS. 9A-9C are images showing Fc exposure of RBC-PEG-SpA-antibody conjugates. Exposure of the Fc region of antibodies coupled to RBC surfaces through SpA binding or direct conjugation was investigated by incubating modified RBCs with ALEXA FLUOR® 488 labeled SpA. Cells were washed before confocal imaging. FIG. 9A includes representative images of RBCs incubated with ALEXA FLUOR®-SpA. FIG. 9B includes representative images of RBC-PEG-IgG conjugates after ALEXA FLUOR®-SpA binding. FIG. 9C includes representative images of RBC-PEG-SpA-IgG conjugates after ALEXA FLUOR®-SpA binding. Scale bars, 20 μm.

FIGS. 10A-10H show in vitro long-term stability of the RBC-PEG-SpA-ALEXA FLUOR® 488-Fc constructs. To monitor the long-term stability of the conjugates, RBC constructs were prepared by exposing the conjugates to ALEXA FLUOR® 488-Fc for 1.5 hours while rotating. Samples were spun down and washed prior to imaging. FIG. 10A includes representative images of unmodified RBCs on day 0. FIGS. 4B-4F show representative images of RBC constructs on day 0 (FIG. 10B), day 8 (FIG. 10C), day 14 (FIG. 10D), day 21 (FIG. 10E), and day 44 (FIG. 10F), respectively. FIG. 10G includes representative images of unmodified RBCs on day 65. FIG. 10H includes representative images of RBC constructs on day 65. Fluorescent images were captured after washing on a Zeiss LSM 880 Meta FCS confocal microscope at 63×. Brightness was adjusted 23% for display purposes. Scale bars, 20 μm.

FIGS. 11A and 11B are graphs plotting in vitro stability of the RBC-PEG-SpA-ALEXA FLUOR® 488-Fc constructs in various media to monitor competitive antibody binding. To monitor the stability of the conjugates, the RBC constructs were exposed to ALEXA FLUOR® 488-Fc for 1.5 hours while rotating and then exposed to different media. FIG. 11A is a graph plotting normalized mean fluorescence intensity of RBC constructs in 50% human serum (diluted half with PBS; squares) or 100% human serum (circles) over 3 days at room temperature with mild rotation. FIG. 11B is a graph plotting normalized mean fluorescence intensity of RBC constructs in AS-3 solution (pH 5.5; circles) or AS-3 solution with 50% human serum (squares) over 43 days at 4° C.

FIGS. 12A and 12B show in vitro stability of the RBC-PEG-SpA-ALEXA FLUOR® 488-Fc constructs in various media, as determined by flow cytometry. Representative scatter plots from flow cytometry are shown, where the y-axis is due to side scattering and the x-axis is the fluorescence intensity from the ALEXA FLUOR® 488-Fc fragment for RBC constructs in acidic (pH 5.5) AS-3 solution on days 0-43 (FIG. 12A), and in AS-3 solution with 50% human serum on days 0-42 (FIG. 12B). Strong and unchanged fluorescence intensities over time indicated that the antibody remained bound to SpA on the RBC surface even at lower pH for 43 days and also in the presence of competing antibodies for at least 42 days.

FIGS. 13A-13C show deformability of RBC-PEG-SpA-IgG constructs at low modification density. FIG. 13A is a graph plotting elongation index versus shear stress plots for RBC constructs synthesized using the indicated synthetic approaches. FIG. 13B is a graph plotting maximum elongation index for the various RBC constructs. FIG. 13C is a graph plotting shear stress at half-max of the various RBC constructs. Modified RBCs deformed similar to unmodified RBCs (control), except for the click chemistry approach, which resulted in less flexible RBCs. Values are mean±standard deviation (*P<0.05; **P<0.01; ***P<0.001 vs. unmodified RBC control).

FIG. 14 is a graph plotting the percentage of phosphatidylserine-exposing RBCs after low, medium, and high-density converted thiol modification, measured by Annexin V-ALEXA FLUOR® 488 binding to phosphatidylserine. Native RBCs oxidized with 0.2 mM CuSO₄/2.5 mM L-ascorbic acid for one hour were used as a positive control for induction of phosphatidylserine.

FIG. 15 is a graph plotting the percentage of hemolysis of RBCs after low, medium, and high-density converted thiol modification, placed under continuous mechanical stress for 1, 4, or 8 hrs. RBC hemolysis from suspension in 5 mM sodium phosphate buffer, pH 8 was recorded as 100% RBC hemolysis.

FIG. 16 is a tertiary-based structure prediction (Carmali et al., ACS Biomater Sci Eng 3:2086-2097, 2017) (PDB: 4WWI) of the C binding domain of SpA (left) in complex with the Fc fragment of human IgG (right), to determine which amino groups were reactive and to ascertain their relative rates. Lysine 58 was predicted to be fast reacting, lysines 4, 7, 35, and 42 were predicted to be slower reacting, and lysines 49 and 50 were predicted to be non-reacting.

FIG. 17 is a reaction scheme depicting hydrolysis (I) and aminolysis (II) of methoxy polyethylene glycol acetic acid N-succinimidyl ester.

FIGS. 18A-18C show deformability of RBC-PEG-SpA-IgG constructs at high modification density. FIG. 18A is a graph plotting elongation index versus shear stress plots of RBC constructs synthesized via different synthetic approaches. FIG. 18B is a graph plotting the maximum elongation index for the various RBC constructs. FIG. 18C is a graph plotting shear stress at half-max of the various RBC constructs. The Bi-NHS chemistry approach at high density yielded constructs that deformed similarly to unmodified RBCs. Values were means±standard deviations (*P<0.05; **P<0.01; ***P<0.001 versus unmodified RBC control).

FIG. 19 shows survival of fluorescently labeled rRBCs in rats. RBCs were modified with ALEXA FLUOR® 647-NHS by reacting the dye with RBCs for 1 hour, with rotating. Samples were spun down and washed prior to injection. The first blood samples were collected 5 minutes after transfusion. Subsequent blood samples were collected every couple of hours up to 14 days. FIG. 19 includes representative images of blood samples taken over time. Fluorescent images were captured on a Zeiss LSM 880 Meta FCS confocal microscope at 40×. Brightness was adjusted to 40% for display purposes. Scale bars, 20 μm.

### DETAILED DESCRIPTION

This document provides erythrocyte constructs in which RBCs are coupled to a linker (e.g., a synthetic polymer) and a biomacromolecule (e.g., a polypeptide, nucleic acid, lipid, or polysaccharide). Studies described elsewhere have shown that erythrocyte membrane antigens can be masked by covalent reaction of PEG with the RBC membrane (Chapanian et al., Biomaterials 33:3047-3057, 2012). Such RBC-polymer conjugates can be stable, functional, biocompatible and long-lived. For example, covalent modification of the RBC surface with polymers such as PEG or methoxy-PEG (mPEG) resulted in increased circulation half-life of the modified cells (Blackall et al., Blood 97:551-556, 2001; Murad et al., Blood 93:2121-2127, 1999; and Scott et al., Proc Natl Acad Sci USA 94:7566-7571, 1997). PEGylation not only prevented antibody recognition but also blocked invasion by the malaria parasite (Blackall et al., supra). Murine RBCs modified with mPEG and injected back into donor mice showed normal in vivo survival of about fifty days (Murad et al., supra). Additionally, sheep RBCs modified with mPEG and injected into mice had a significantly longer half-life in circulation compared to unmodified sheep RBCs (Scott et al., supra). PEGylated RBCs were also shown to have reduced aggregation and low shear blood viscosity compared to unmodified cells (Armstrong et al., Am J Hematol 56:26-28, 1997). It is noted, however, that over-modification by affinity binding to structurally important targets can alter crucial features and activities of the RBC such as deformability, inhibition of complement binding and activation, and evasion of phagocytosis (Villa et al., supra; Paulitschke et al., Blood 86:342-348, 1995; Chasis et al., J Clin Invest 75:1919-1926, 1985; and Straat et al., Transfusion Med Hemother 39:353-360, 2012).

The present document provides methods for attaching polymers and polypeptides (or other biomacromolecules) to RBCs. The resulting RBC conjugates are stable and can provide high-density modification of the RBC surface while maintaining the deformability and function of the RBCs and attached biomacromolecules. In some cases, the methods provided herein can be used to generate conjugates in which antibodies are attached to RBCs via polymer linkers. RBC-antibody conjugates can be used to treat disease by, for example, scavenging biological molecules from the circulation. Immunoglobulin-based therapeutics have been spectacularly successful in treating a wide range of diseases. The challenges of therapeutic immunoglobulin delivery include their relatively short lifetimes in vivo that require regular injection of the drug, the unpredictable immunological consequences resulting from the available Fc region of the antibody-antigen complex, and the development of anti-drug antibodies. The methods and materials provided herein can address one or more of these issues.

In general, this document provides membrane-engineered erythrocytes that can be chemically synthesized to display a suitable biomacromolecule on their outer surface. In some cases, when the biomacromolecule is an antibody, the antibody can be displayed in a manner that masks its Fc region. As compared to previous antibody therapeutics, the methods and materials described herein effectively shift the location of the antibody from the plasma to a cell surface, which can decrease the required therapeutic dose, decrease the required delivery frequency, and diminish the negative consequences that may result from accumulation of plasma-borne antibody-antigen complexes. The Examples below demonstrate that an antibody (e.g., anti-TNF) can be stably and functionally bound to a long-lived, plasma-borne cell, without sacrificing antigen binding capacity and cellular function. The RBC-antibody complexes provide an inert, biocompatible, immune-privileged and long-lived nanoparticle in the blood compartment while hiding the antibody Fc regions, and are thus an effective mechanism for removing target antigens from the circulation. In some embodiments, for example, RBCs can be coupled to anti-TNF antibodies, in order to reduce the level of TNFα in the blood stream.

The cells used in the methods and materials described herein therefore have the capacity to display immunoglobulins or other biomacromolecules, but from a membrane that is not rapidly turned over. Most human cells turn over their membrane proteins over the course of days, but red blood cells (RBCs) are enucleated and as such, they are not in a position to replace membrane proteins regularly, if at all. Since polymers can covalently react with cell membranes and tissues (Burchenal et al., J Thrombosis Thrombolysis 13:27-33, 2002; Panza et al., Biomaterials 21:1155-64, 2000; Clafshenkel et al., Plos One 11(6): e0157641, 2016; and D'Souza et al., Biomaterials 35:9447-9458, 2014), erythrocytes were selected and developed as biocompatible, long-lived, and immune-privileged circulatory drug delivery particles (Muzykantov, Exp Opin Drug Deliv 7:403-27, 2010; and Villa et al., supra), as described herein. The chemistry-based methods provided herein are faster, simpler, and more easily fine-tuned than other (e.g., molecular biology) approaches.

The biologic impact of covalently coupling polymers to an RBC membrane is a function of the number of modification sites. Studies of murine RBCs that used biotinylation to tag the cells found a density of 6×10⁵ biotin molecules/cell (Gimsa and Ried, Mol Membrane Biol 12:247-254, 1995), and the binding density of avidin to biotin-phosphatidylethanolamine in the erythrocyte membrane was approximated at 5×10⁵ molecules/RBC (Muzykantov et al., J Immunol Meth 158:183-190, 1993). Reaction of NHS-poly-dimethyl acrylamide with human RBCs also showed a binding density of 5×10⁵ molecules/cell. Until recently, the reaction density of PEG on RBCs was only characterized by the concentration of reactive PEG in the binding reaction that caused the least damage (Hashemi-Najafabadi et al., Bioconj Chem 17:1288-1293, 2006; and d'Almeida et al., Transfusion Med 10:291-303, 2000). The density has since been shown to be 1.5×10⁵ molecules/cell for a hyperbranched PEG molecule (Chapanian et al., supra). A single RBC can thus carry a payload array of about 100,000 molecules without any significant biologic impact on the cell. Chemistries developed to couple polymers to the surface of RBCs for RBC-drug conjugates have included coupling to amine (Clafshenkel et al., supra), cysteine (McCombs and Owen, Aaps J 17:339-351, 2015; and Broyer et al., Chem Commun 47:2212-2226, 2011), and other functional groups (Besanceney-Webler et al., Angewandte Chemie—Int Ed 50:8051-8056, 2011; Uttamapinant et al., Angewandte Chemie—Int Ed 51:5852-5856, 2012; and Li et al., Chem Sci 8:2107-2114, 2017).

The chemistry described herein allows for the controllable and covalent attachment of up to 100,000 molecules to the exterior membrane of each RBC. The coupling reaction was designed to work under physiological conditions, and purification of the modified cells is straightforward.

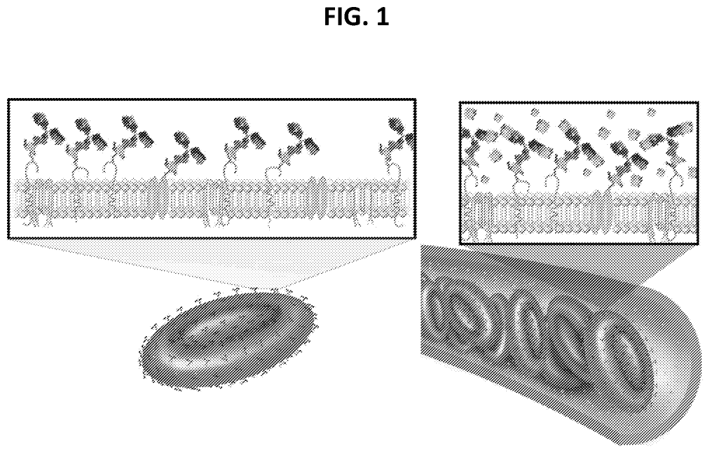

In some cases, as described herein, the erythrocyte constructs can have Staphylococcus aureus protein A (SpA) coupled to the surface of RBCs via the polymer linker. SpA is a protein bound to the outer surface of staphylococcal cells, and it also can bind strongly to the Fc region of IgG The ability to bind antibodies by their Fc regions can orient the antibodies with their antigen binding sites facing out, thus preventing recognition of the bacteria by neutrophils. The binding site of SpA on the IgG-Fc overlaps the binding site of FcRn (DeLano et al., Science 287:1279-1283, 2000) and neutrophil receptors (Graille et al., Proc Natl Acad Sci USA 97:5399-5404, 2000). SpA is itself an anti-inflammatory molecule that can be used in drug development (Bernton and Haughey, Basic Clin Pharmacol Toxicol 115:448-455, 2014). When the attached molecule is SpA, the Fc region of antibodies (e.g., anti-TNFα antibodies) can be bound to the RBC-SpA arrays in vitro, establishing a facile two-step assembly of a long-lived, biocompatible and functional RBC for removal of biological molecules (e.g., TNF) from the circulation, as illustrated in FIG. 1.

Thus, described herein are materials and methods for the chemical synthesis and use of RBC complexes, including RBC-SpA complexes, that have the potential to bind and present any antibody within plasma. As described in the Examples herein, erythrocytes with covalently attached SpA-mediated arrays of anti-TNFα were shown to remove TNFα from physiological buffer. Up to 37% of rat or human anti-TNFα was biologically available for TNF binding in the SpA-mediated antibody arrays on erythrocytes in vitro. In a short-term in vivo experiment, SpA-mediated antibody arrays on the surface of RBCs were detectable after injection into rats. The long-term in vivo efficacy of chemically membrane-engineered RBCs to remove antigens like TNF from serum can be evaluated in one or more animal models.

In general, the erythrocyte constructs provided herein include at least three components: an RBC, a synthetic polymer with at least two coupling moieties, and one or more biomacromolecules. In some cases, the constructs each include two or more biomacromolecules, where a first biomacromolecule is SpA and a second is an antibody that interacts with the SpA.

Erythrocytes can be obtained from any appropriate organism and used to generate the constructs provided herein. For example, RBCs can be obtained from a mammal (e.g., a human, non-human primate, rat, mouse, cat, dog, horse, cow, sheep, pig, goat, or rabbit). Once collected, the outer surface of the RBCs can be modified by coupling a polymer or another compound as described herein (e.g., Traut's reagent or a Click reagent) to a functional group thereon. For example, amino groups or thiol groups on the erythrocyte surface can be targeted for attachment of a polymer, or amino groups on the erythrocyte surface can be targeted for modification prior to polymer attachment.

Any suitable polymer can be used in the constructs and methods described herein. In general, the final erythrocyte constructs include a RBC coupled to a biomacromolecule via a polymer that includes at least two coupling moieties. It is noted, however, that the polymer in the final constructs may result from the interaction of two “partial polymers,” such as Click reagents.

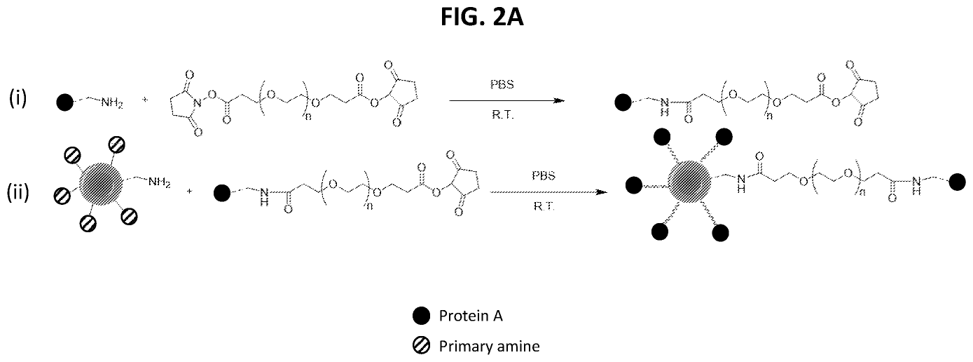

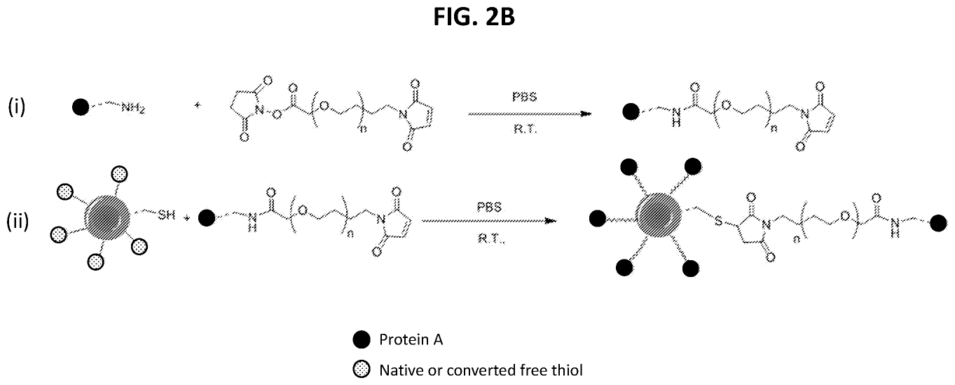

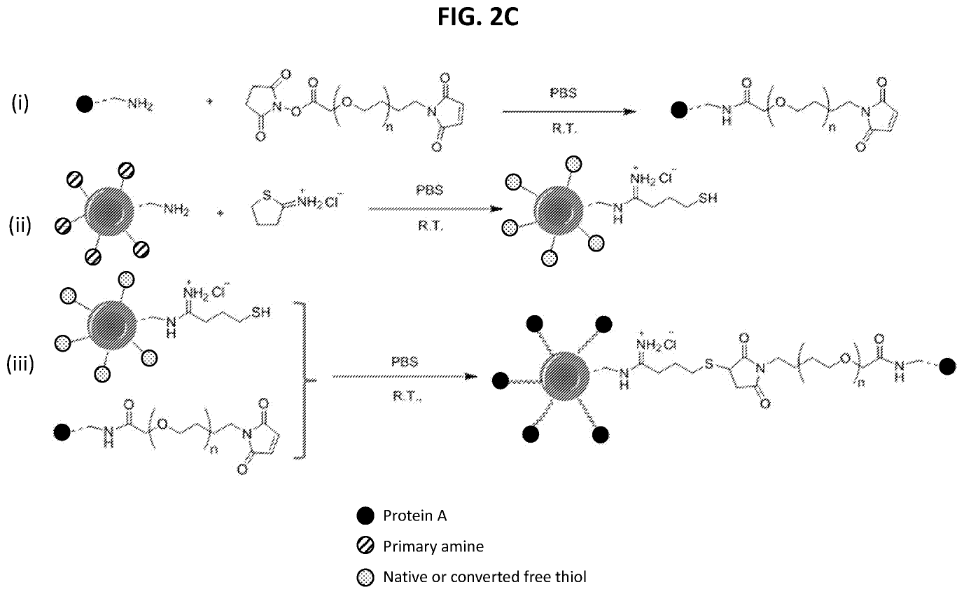

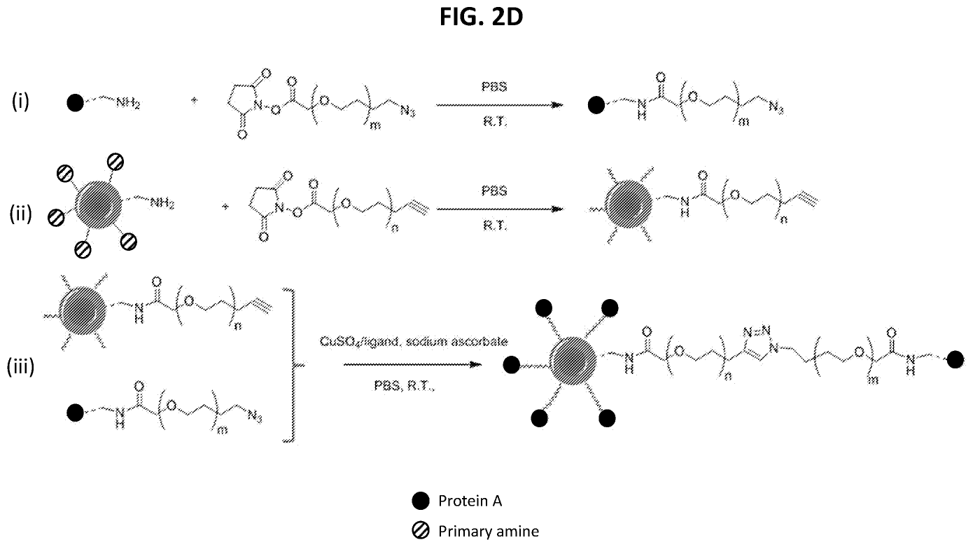

In some cases, as noted above, the polymers can be coupled to amino groups or thiol groups on the RBC surface. In some cases, the polymers also can be coupled to the biomacromolecules via amino or thiol groups. Any appropriate moiety can be used to couple a polymer to an amino group or a thiol group. Suitable moieties for coupling to amino groups include, without limitation, NHS, succinimidyl derivatives, propionaldehyde, butryraldehyde, and benzotriazole. Suitable moieties for coupling to thiol groups include, without limitation, MAL, MAL derivatives, vinyl sulfone, and thiol derivatives. As set forth in the particular reaction schemes described herein (and illustrated in FIGS. 2A-2D), in some cases, a polymer can include two or more moieties for coupling to amino groups (e.g., two or more NHS moieties), two or more moieties for coupling to thiol groups (e.g., two or more MAL moieties), or at least one moiety for coupling to an amino group and at least one moiety for coupling to a thiol group (e.g., an NHS moiety and a MAL moiety). It is noted, however, that any suitable combination of coupling moieties can be used.

Any appropriate polymer can be used in the constructs provided herein. In some cases, PEG can be used, as described in the Examples below. Other examples of suitable polymers include, without limitation, other polyalkylene glycols, such as polypropylene glycol, ethylene glycol and propylene glycol copolymers, polyvinylpyrrolidone, polyhydroxyalkylmethacrylamide, polyhydroxyalkylmethacrylate, poly-α-hydroxy acid, polyvinyl alcohol, and any combination thereof. The polymer can be a homopolymer or copolymer (e.g., an alternating copolymer, random copolymer, block copolymer, etc., made up of monomers (e.g., individual units of any of the polymers listed herein). The polymer can be linear or branched, or may include multiple branches or arms. Moreover, the polymer can have any appropriate molecular weight. For example, the polymer can have a molecular weight between about 100 Daltons to about 100,000 Daltons (e.g., about 500 Daltons to about 75,000 Daltons, about 1,000 Daltons to about 50,000 Daltons, about 2,000 Daltons to about 25,000 Daltons, about 3,000 Daltons to about 20,000 Daltons, about 5,000 Daltons to about 15,000 Daltons, or about 7,000 Daltons to about 10,000 Daltons). It is noted that in general, the polymer is synthetic (also referred to as “non-naturally occurring”), meaning that the polymer in its entirety is not found in nature. It is to be noted that although a non-naturally occurring polymer can include one or more subunits or portions of subunits that are naturally occurring, the polymer in its entirety is not found in nature.

The erythrocytes in the conjugates provided herein can be coupled, via the polymer, to any suitable biomacromolecule. A “biomacromolecule” is a biological polymer, such as a polypeptide, a nucleic acid, a carbohydrate, or a lipid, that is made up of monomers linked together.

Thus, in some cases, the biomacromolecule is a nucleic acid. As used herein, the term “nucleic acid” refers to RNA and DNA, including cDNA, genomic DNA, synthetic (e.g., chemically synthesized) DNA, and DNA (or RNA) containing nucleic acid analogs. Nucleic acids can have any three-dimensional structure, and can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In some cases, the biomacromolecule is a polypeptide. The term “polypeptide,” as used herein, refers to a compound of two or more subunit amino acids, regardless of post-translational modification (e.g., phosphorylation or glycosylation). In some cases, a polypeptide can be a full-length, functional protein. In other cases, a polypeptide can be a fragment of a full-length protein, or a variant of a full length protein that may or may not function in the manner of the corresponding non-variant protein. The subunits of a polypeptide can be linked by peptide bonds or other bonds such as, for example, ester or ether bonds. “Amino acid” refers to either natural and/or unnatural or synthetic amino acids, including D/L optical isomers.

When the biomacromolecule is a polypeptide, any appropriate polypeptide can be used. These include, without limitation, antibodies (e.g., antibodies that can bind specifically to biological molecules such as, without limitation, TNFα, thereby scavenging them and removing them from the circulation), enzymes, antigens, and other therapeutic polypeptides. In some cases, the biomacromolecule can be SpA, and the RBC-polymer-SpA conjugate can then be coupled to an antibody via interaction of SpA with the antibody's Fc region. When administered into the circulatory system of a subject (e.g., a human), the RBC-polymer-SpA-Ab complex can present the antibodies to the surrounding circulatory environment, and can effectively remove from the circulation molecules to which the antibody binds.

As used herein, the term “antibody” refers to any immunoglobulin or antibody (e.g., human, hamster, or mouse antibody), and any derivative or conjugate thereof, that specifically binds to an antigen. Non-limiting examples of antibodies include monoclonal antibodies, polyclonal antibodies, humanized antibodies, multi-specific antibodies (e.g., bispecific antibodies), single-chain antibodies (e.g., single-domain antibodies, camelid antibodies, and cartilaginous fish antibodies), chimeric antibodies, feline antibodies, and felinized antibodies. The term “antibody” also includes antibody derivatives and conjugates (e.g., an antibody conjugated to a stabilizing protein, a detectable moiety, or a therapeutic agent). Antigen binding fragments of antibodies also can be used in the constructs and methods provided herein. An “antigen binding fragment” is any portion of a full-length antibody that contains at least one variable domain (e.g., a variable domain of a heavy or light chain immunoglobulin) that is capable of specifically binding to an antigen. Non-limiting examples of antibody fragments include Fab, Fab′, F(ab′)2, and Fv fragments, diabodies, linear antibodies, and multi-specific antibodies formed from antibody fragments.

An Fv fragment is the minimum antibody fragment that contains a complete antigen recognition and binding site. This region consists of a dimer of one heavy chain variable domain and one light chain variable domain in tight, non-covalent association. It is in this configuration that the three complementary determining regions (CDRs) of each variable domain interact to define an antigen binding site on the surface of the V_{H}-V_{L} dimer. A “complementary determining region” or “CDR” is a region within an immunoglobulin (a heavy or light chain immunoglobulin) that forms part of an antigen binding site in an antibody or antigen binding fragment thereof. Heavy chain and light chain immunoglobulins each contain three CDRs, referred to as CDR1, CDR2, and CDR3. In any antibody or antigen binding fragment, the three CDRs from the heavy chain immunoglobulin and the three CDRs from the light chain immunoglobulin together form an antigen binding site in the antibody or antigen binding fragment thereof. The Kabat Database is one system used in the art to number CDR sequences present in a light chain immunoglobulin or a heavy chain immunoglobulin.

Collectively, the six CDR's confer antigen binding specificity to the antibody. However, even a single variable domain (or half of an Fv comprising only three CDR's specific for an antigen) has the ability to recognize and bind the antigen, although usually at a lower affinity than the entire binding site. The “Fab fragment” also contains the constant domain of the light chain and the first constant domain (CH1) of the heavy chain. The “Fab fragment” differs from the “Fab′ fragment” by the addition of a few residues at the carboxy terminus of the heavy chain C_{H}1 domain, including one or more cysteines from the antibody hinge region. The “F(ab′)2 fragment” originally is produced as a pair of “Fab′ fragments” which have hinge cysteines between them. Methods of preparing such antibody fragments include, without limitation, papain or pepsin digestion.

An antibody can be of the IgA-, IgD-, IgE, IgG- or IgM-type, including IgG- or IgM-types such as, without limitation, IgG1-, IgG2-, IgG3-, IgG4-, IgM1- and IgM2-types. For example, in some cases, the antibody is of the IgG1-, IgG2- or IgG4-type.

In some embodiments, antibodies can be fully human or humanized antibodies. By “human antibody” is meant an antibody that is encoded by a nucleic acid (e.g., a rearranged human immunoglobulin heavy or light chain locus) present in the genome of a human. In some embodiments, a human antibody can be produced in a human cell culture (e.g., feline hybridoma cells). In some embodiments, a human antibody can be produced in a non-human cell (e.g., a mouse or hamster cell line). In some cases, a human antibody can be produced in a bacterial or yeast cell.

The term “humanized antibody” refers to a human antibody that contains minimal sequence derived from non-human (e.g., mouse, hamster, rat, rabbit, or goat) immunoglobulin. Humanized antibodies generally are chimeric or mutant monoclonal antibodies from mouse, rat, hamster, rabbit or other species, bearing human constant and/or variable region domains or specific changes. In non-limiting examples, humanized antibodies are human antibodies (recipient antibody) in which hypervariable region (HVR) residues of the recipient antibody are replaced by HVR residues from a non-human species (donor) antibody, such as a mouse, rat, rabbit, or goat antibody having the desired specificity, affinity, and capacity. In some embodiments, Fv framework residues of the human immunoglobulin can be replaced by corresponding non-human residues. In some embodiments, humanized antibodies can contain residues that are not found in the recipient antibody or in the donor antibody. Such modifications can be made to refine antibody performance, for example.

In some embodiments, a humanized antibody can contain substantially all of at least one, and typically two, variable domains, in which all or substantially all of the hypervariable loops (CDRs) correspond to those of a non-human immunoglobulin, while all or substantially all of the framework regions are those of a human immunoglobulin sequence. A humanized antibody also can contain at least a portion of an immunoglobulin constant (Fc) region, typically that of a human immunoglobulin.

As used herein, the term “single-domain antibody” refers to a polypeptide that contains one camelid VHH or at least one cartilaginous fish Ig-NAR domain that is capable of specifically binding to an antigen. Non-limiting examples of single-domain antibodies are described, for example, in U.S. Publication No. 2010/0092470.

An antibody or antigen binding fragment thereof “specifically binds” to a particular antigen (e.g., TNFα) when it binds to that antigen in a sample, and does not recognize and bind, or recognizes and binds to a lesser extent, other molecules in the sample. In some cases, an antibody or an antigen binding fragment can selectively bind to an epitope with an affinity (KD) of about, for example, 1×10⁻⁶M or less (e.g., about 1×10⁻⁷ M, about 1×10⁻⁸ M, about 1×10⁻⁹ M, or less) in phosphate buffered saline. The ability of an antibody or antigen binding fragment to specifically bind a protein epitope can be determined using any appropriate method, such as binding to an immobilized substrate coupled to the target epitope with detection using an ELISA method, binding to the target epitope on live cells with detection by flow cytometry, or binding to an immobilized target epitope by surface plasmon resonance.

Other polypeptides that can be included in the constructs provided herein include, without limitation, enzymes that may be missing or produced at lower than normal levels in a subject to be treated, or enzymes that can degrade toxic metabolites that can accumulate in the blood stream.

In some cases, a polypeptide included in an erythrocyte construct provided herein can include without limitation, SpA, Protein G, calcitonin, co-stimulatory molecules, erythropoietin, Factor VI, Factor IX, cyclosporin, granulocyte colony stimulating factor, thrombopoietin, alpha-1 proteinase inhibitor, granulocyte macrophage colony stimulating factor, human growth hormone, growth hormone releasing hormone, interferons, interleukins, interleukin receptors, interleukin receptor antagonists, insulin, somatostatin, vasopressin, follicle stimulating hormone, insulin-like growth factor, macrophage colony stimulating factor, nerve growth factor, tissue growth factors, keratinocyte growth factor, tumor necrosis factor, endothelial growth factors, alpha-1 antitrypsin, or a peptide antigen. In some cases, the polypeptide can be an enzyme (e.g., a lyase, a uricase, a synthase, an esterase, a lipase, a hydrolase, an aminase, an oxidoreductase, a hydrogenase, lysozyme, a transaminase, an asparaginase, a amylase, a peptidase, or a protease). It is to be noted that a polypeptide used in the erythrocyte construct as provided herein can be synthetic, native, glycosylated, or unglycosylated, or can be a biologically active fragment or analog of a native protein.

This document also provides methods for synthesizing RBC-polymer-biomacromolecule complexes. The methods described herein demonstrate the breadth of chemical membrane engineering. Examples include coupling an N-hydrosuccinidyl ester-functionalized homobifunctional polymer (e.g., NHS-PEG-NHS) to amino groups on RBCs and on a population of biomacromolecules, coupling a heterobifunctional maleimide-polymer-NHS ester (e.g., MAL-PEG-NHS) to thiol groups naturally present or engineered onto the RBC surface (e.g., using Traut's reagent to effectively convert amino groups to thiols) and to amino groups on a population of biomacromolecules, or coupling a heterobifunctional azido-polymer-NHS ester (e.g., N3-PEG-NHS) and an alkyne-polymer-NHS ester (e.g., Alk-PEG-NHS) to amino groups on one or the other of a population of RBCs and a population of biomacromolecules, and then coupling the azido group to the alkyne. As described in the Examples below, when SpA is used, RBC-polymer-SpA complexes can be synthesized within minutes, and over 10⁵ antibodies per RBC (human and rat) can be attached to the accessible erythrocyte-bound SpA molecules. The RBC-SpA-Ab arrays described in the Examples were shown to be stable in vitro for more than 60 days in PBS, and for more than 8 days in serum containing medium or whole serum.

Thus, in some embodiments, the methods provided herein can include reacting a polymer with amino groups on a biomacromolecule (e.g., a polypeptide, such as an enzyme or SpA), and with amino groups on the RBC surface. Suitable polymers for use in such embodiments therefore can have at least first and second moieties capable of reacting with amino groups (e.g., NHS or other suitable moieties). In some cases, the polymer can be combined with the biomacromolecule to generate a polymer-biomacromolecule conjugate, and the conjugate can then be combined with RBCs to generate the RBC construct (see, e.g., FIG. 2A). In some cases, the reactions can be carried out in a single reaction vessel (e.g., in a single, multi-step reaction), either by simultaneous addition of the polymer, biomacromolecule, and RBCs to the vessel, or by generation of the polymer-biomacromolecule conjugate followed by addition of the RBCs to the vessel. In some cases, the reactions can be carried out in separate reaction vessels. A particular example of a method for synthesizing erythrocyte constructs in which SpA is coupled to RBCs via conjugation of a polymer (NHS-PEG-NHS) to amino groups is described in the Examples below.

In some embodiments, the methods provided herein can include reacting a polymer with amino groups on a biomacromolecule (e.g., a polypeptide, such as an enzyme or SpA), and with thiol groups on the RBC surface. Suitable polymers for use in such embodiments therefore can have at least a first moiety (e.g., NHS) capable of reacting with amino groups and a second moiety (e.g., MAL) capable of reacting with thiol groups. In some cases, the polymer can be combined with the biomacromolecule to generate a polymer-biomacromolecule conjugate, and the conjugate can then be combined with RBCs to generate the RBC construct (see, e.g., FIG. 2B). The reactions can be carried out in a single reaction vessel (e.g., in a single, multi-step reaction) or in separate reaction vessels. A particular example of a method for synthesizing erythrocyte constructs in which SpA is coupled to RBCs via conjugation of a polymer (MAL-PEG-NHS) to amino groups on the SpA and free thiol groups on the RBCs is described in the Examples below.

In some embodiments, the methods provided herein can include reacting a polymer with amino groups on a biomacromolecule (e.g., a polypeptide, such as an enzyme or SpA), and with thiol groups that are engineering onto the RBC surface essentially by conversion of amino groups to thiol groups. Suitable polymers for use in such embodiments therefore can have at least a first moiety (e.g., NHS) capable of reacting with amino groups and a second moiety (e.g., MAL) capable of reacting with thiol groups. In such embodiments, RBCs are treated with Traut's reagent to convert free amino groups to thiol groups, and in a separate reaction, the polymer can be combined with the biomacromolecule to generate a polymer-biomacromolecule conjugate. The polymer-biomacromolecule conjugate can then be combined with the thiol-engineered RBCs to generate the RBC construct (see, e.g., FIG. 2C). A particular example of a method for synthesizing erythrocyte constructs in which SpA is coupled to RBCs via conjugation of a polymer (MAL-PEG-NHS) to amino groups on the SpA and amino groups converted to thiol groups on the RBCs is described in the Examples below.

In some embodiments, the methods provided herein can include using “Click” chemistry to react a first Click polymer reagent with amino groups on a biomacromolecule (e.g., a polypeptide, such as an enzyme or SpA), and a second Click polymer reagent with amino groups on the RBC surface. Suitable Click polymer reagents for use in such embodiments therefore can include moieties (e.g., NHS groups) capable of reacting with amino groups. In general, the first Click polymer reagent can be combined with the biomacromolecule to generate a polymer-biomacromolecule-Click conjugate, the second Click polymer reagent can be combined with RBCs to generate a RBC-polymer-Click conjugate, and the conjugates can then be combined to generate the RBC construct (see, e.g., FIG. 2D). A particular example of a method for synthesizing erythrocyte constructs in which SpA is coupled to RBCs via conjugation of a first Click polymer reagent (N3-PEG-NHS) to amino groups on the SpA and conjugation of a second Click polymer reagent (Alk-PEG-NHS) to amino groups on the RBCs is described in the Examples below.

The methods provided herein can yield RBC constructs coupled to polymers and biomacromolecules at a density of about 10⁴ to about 10⁸ polymer chains/biomacromolecules per cell. In some cases, “low” modification density can refer to about 10⁴ to about 10⁵ (e.g., about 1×10⁴ to about 2×10⁴, about 2×10⁴ to about 4×10⁴, about 3×10⁴ to about 5×10⁴, about 5×10⁴ to about 8×10⁴, or about 7×10⁴ to about 1×10⁵) polymer chains/biomacromolecules. In some cases, In some cases, “medium” modification density can refer to about 10⁵ to about 10⁶ (e.g., about 1×10⁵ to about 2×10⁵, about 2×10⁵ to about 4×10⁵, about 3×10⁵ to about 5×10⁵, about 5×10⁵ to about 8×10⁵, or about 7×10⁵ to about 1×10⁶) polymer chains/biomacromolecules. In some cases, “high” modification density can refer to about 10⁶ to about 10⁸ (e.g., about 1×10⁶ to about 2×10⁶, about 2×10⁶ to about 4×10⁶, about 3×10⁶ to about 5×10⁶, about 5×10⁶ to about 8×10⁶, about 7×10⁶ to about 1×10⁷, about 1×10⁷ to about 2×10⁷, about 2×10⁷ to about 4×10⁷, about 3×10⁷ to about 5×10⁷, about 5×10⁷ to about 8×10⁷, or about 7×10⁷ to about 1×10⁸) polymer chains/biomacromolecules.

This document also provides methods for treating mammals (e.g., humans, non-human primates, rats, mice, cats, dogs, horses, cows, sheep, pigs, goats, or rabbits) by administration of one or more erythrocyte constructs provided herein. The mammal can be, for example, a mammal in need of treatment by enzyme replacement therapy (e.g., for treatment of enzyme deficiency), antigen therapy (e.g., for treatment of an autoimmune disorder), or antibody therapy (e.g., for removing toxins from the blood). The methods can include administering an RBC construct as provided herein to a mammal in need thereof. The construct typically can be administered directly into the bloodstream of the recipient, e.g., by intravenous injection or infusion. Any suitable amount

In some cases, one or more RBC constructs can be administered to a mammal once or multiple times over a period of time ranging from days to months. In some cases, one or more RBC constructs can be formulated into a pharmaceutically acceptable composition for administration to a mammal that has been identified as being in need of treatment for a condition such as, without limitation, an enzyme deficiency or an autoimmune disorder. For example, a therapeutically effective amount of a RBC construct can be formulated together with one or more pharmaceutically acceptable carriers (additives) and/or diluents. Pharmaceutically acceptable carriers, fillers, and vehicles that may be used in a pharmaceutical composition include, without limitation, sterile water, ion exchangers, alumina, aluminum stearate, lecithin, serum proteins, such as human serum albumin, buffer substances such as phosphates, glycine, sorbic acid, potassium sorbate, salts or electrolytes, disodium hydrogen phosphate, potassium hydrogen phosphate, sodium chloride, and zinc salts. In some cases, the compositions can include aqueous and/or non-aqueous sterile injection solutions that may contain anti-oxidants, buffers, bacteriostats, and solutes that render the formulation isotonic with the blood of the intended recipient.

Effective doses can vary depending on the severity of the mammal's condition, the age and general health condition of the subject, excipient usage, the possibility of co-usage with other therapeutic treatments, and the judgment of the treating clinician.

An effective amount of a composition containing RBC constructs can be any amount that reduces a symptom of the recipient mammal's condition, without producing significant toxicity to the mammal. In some cases, for example, about 10⁶ to about 10²¹ or more RBCs (e.g., about 10⁶ to about 10⁹, about 10⁹ to about 10¹², about 10¹² to about 10¹⁵, about 10¹⁵ to about 10¹⁸, about 10¹⁸ to about 10²¹, or more than 10²¹ RBCs) can be administered to an average sized human (e.g., about 75-85 kg human) per administration.

If a particular mammal fails to respond to a particular amount, then the amount of the administered RBC construct(s) can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition may require an increase or decrease in the actual effective amount administered. In some cases, the frequency of administration can be from about once a day to about once a month (e.g., from about once a week to about once every other week). The frequency of administration can remain constant or can be variable during the duration of treatment. A course of treatment with a composition containing one or more RBC constructs can include rest periods. As a non-limiting example, a composition containing a RBC construct can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency. In some cases, the duration of treatment can vary from several days to several months to a year or more. Multiple factors can influence the actual effective duration used for a particular treatment. For example, an effective duration can vary with the frequency of administration, effective amount, use of multiple treatment agents, route of administration, and severity of the condition being treated.

The invention will be further described in the following examples, which do not limit the scope of the invention described in the claims.

### Examples

### Example 1—Materials and Methods

Materials: Polyethylene glycol-bis (N-succinimidyl succinate) (NHS-PEG₅₀₀₀-NHS), maleimide-PEG₅₀₀₀-NHS, azido-PEG₂₀₀₀-NHS, and alkyne-PEG₃₀₀₀-NHS were purchased from Nanocs Inc. (New York, N.Y.). 2-(4-((bis((1-(tert-butyl)-1H-1,2,3-triazol-4-yl) methyl) amino) methyl)-1H-1,2,3-triazol-1-yl) acetic acid (BTTAA) was obtained from Click Chemistry Tools (Scottsdale, Ariz.). Traut's reagent (2-iminothiolane hydrochloride) and Pageruler Plus protein ladder were purchased from Fisher Scientific (Waltham, Mass.). Copper (II) sulfate, sodium ascorbate, and Staphylococcal Protein A were ordered from Sigma Chemical Co. (St Louis, Mo.). Recombinant Staphylococcal Protein A was ordered from ProSpec-Tany TechnoGene Ltd (East Brunswick, N.J.). N-2-bromo-2-methylpropionyl-β-alanine N′-oxysuccinimide ester (NHS-Br) NHS compound was prepared as described elsewhere (Murata et al., Biomacromolecules 14:1919-1926, 2013). Sulfo-Cyanine5 NHS ester, sulfo-Cyanine5 maleimide, and sulfo-Cyanine5 carboxylic acid were purchased from Lumiprobe Corporation (Hunt Valley, Mass.). 2× Laemmli SDS gel loading buffer was obtained from Bio-Rad (Hercules, Calif.). 2-mercaptoethanol was purchased from Sigma (St. Louis, Mo.) and was added to 2× Laemmli gel loading buffer. DiSulfo-Cy5 N-succinimidyl ester (diSulfo-Cy5-NHS) was purchased from Cyandye, LLC (Sunny Isles Beach, Fla.). ALEXA FLUOR® 488 labeled human IgG whole antibodies and Fc fragments were obtained from Jackson ImmunoResearch Laboratories, Inc. (West Grove, Pa.). Dulbecco's phosphate buffered saline (DPBS) without calcium and magnesium and Dead Cell Apoptosis Kits were obtained from Invitrogen (Carlsbad, Calif.). Leukocyte-reduced, packed human RBCs were provided by the Institute of Transfusion Medicine (ITxM, Pittsburgh, Pa.). AS-3 solution was from Boston BioProducts Inc. (Ashland, Mass.). Human serum from human male AB plasma and Penicillin-Streptomycin were purchased from Sigma. All other chemicals and reagents were of analytic grade and used as received.

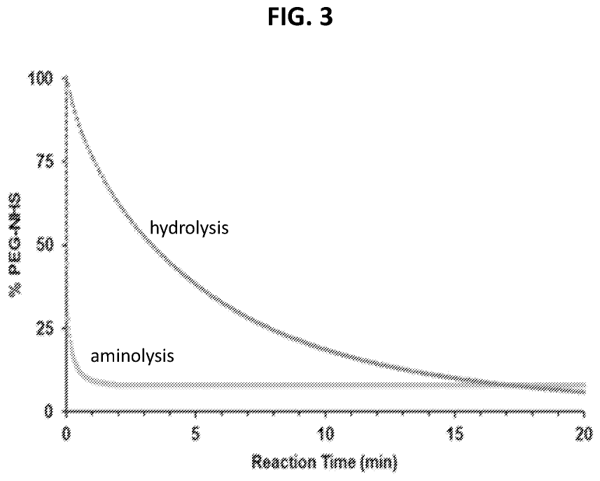

Hydrolysis of methoxy polyethylene glycol acetic acid N-succinimidyl ester (mPEG₅₀₀₀-NHS): A stock solution of 20 mM mPEG₅₀₀₀-NHS was prepared by dissolving 50 mg in 0.5 mL of dimethyl sulfoxide (DMSO). Hydrolysis was performed by mixing a stock solution of mPEG-NHS (100 μL) with phosphate buffered saline, pH 7.4 (900 μL) to give ˜2 mM final concentration of PEG in a quartz cuvette. Reaction progress was measured as the increase in absorbance of the N-hydroxysuccinimide (NHS) ester group at 295 nm over 20 minutes. Measurements were performed in triplicate. Absorbance data were converted into NHS-ester leaving group concentration, with PEG consumption calculated by subtraction of the NHS leaving group concentration from initial PEG-NHS concentration for each time point. Data were plotted and the NHS half-life was determined (FIG. 3).

Aminolysis of mPEG₅₀₀₀-NHS with Na-(Carbobenzyloxy)-L-lysine (Z-Lys-OH): To determine the reaction rate between the mPEG-NHS and amino groups, a stock solution of 35.7 mM Z-Lys-OH was prepared by dissolving 10 mg of the amine compound in 1 mL phosphate buffer, pH 7.4. Z-Lys-OH solution (260 μL) was mixed with phosphate buffer, pH 7.4 (640 μL) to generate a solution with 9.3 mM final concentration of amine in the cuvette (final volume 1 mL). Stock solution (100 μL of 20 mM) of mPEG-NHS was then added and the aminolysis rates were determined by the increase in absorbance of N-hydroxysuccinimide (NHS) ester group at 295 nm, similar to hydrolysis. (FIG. 3).

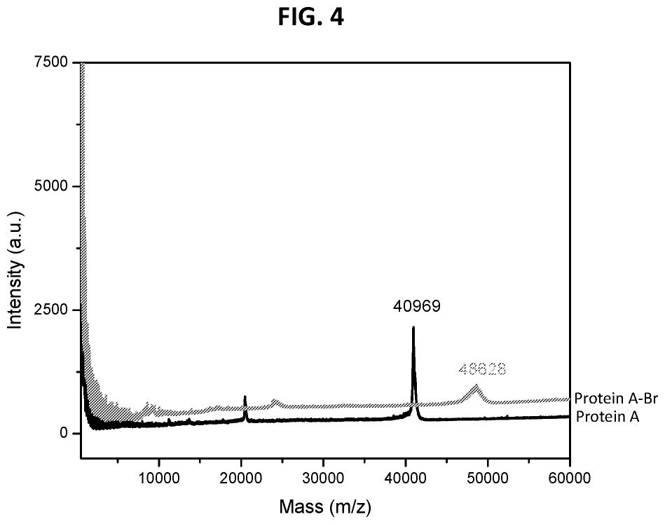

Quantification of accessible primary amines of SpA: To determine how many amino group sites were available on SpA for modification, a small molecule (NHS-Br) was used. 10.0 mg of SpA protein (0.24 μmol) was dissolved in 5 mL of 100 mM sodium phosphate buffer at pH 8.0, and 14.7 mg of NHS-Br (0.044 mmol) was added into the SpA solution. The reaction was stirred at room temperature for 1 hour, after which the mixture was dialyzed against deionized water for 36 hours in a dialysis cassette with MWCO 10.0 kDa. SpA-Br was finally harvested through lyophilization. Reaction of NHS-Br with primary amines on SpA were confirmed by a fluorescamine assay and MALDI-TOF MS (FIG. 4).

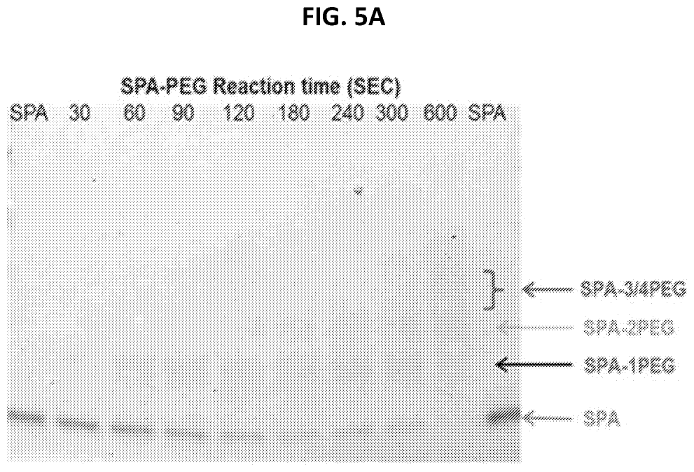

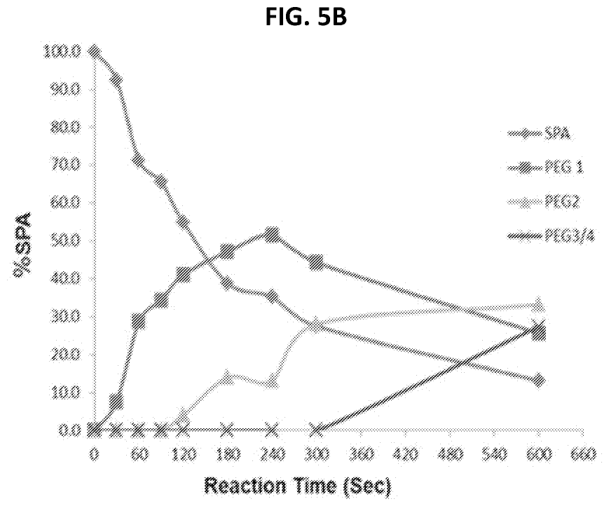

SDS-PAGE gel kinetics of NHS-PEG-SpA conjugate formation: SpA (1.0 mg) was suspended in PBS (1.0 mL at pH 7.4) and added directly to NHS-PEG₅₀₀₀-NHS (1.0 mg). Aliquots (10 μL) were withdrawn at timed intervals and immediately added to 10 μL of 2×SDS-glycine gel loading buffer and placed directly into a boiling water bath for 2 minutes to stop the reaction and denature the protein. Control (time zero) samples were taken prior to mixing with the PEG. 10 μL of each sample was loaded on a 7.5 SDS-PAGE gel (Bio-Rad, Hercules, Calif.). Following electrophoresis, the gel was stained with PAGE-Blue protein stain (Thermo Scientific), imaged, and analyzed with Bio-Rad CHEMIDOC™ Touch Imaging System and Image Lab software (FIGS. 5A and 5B).

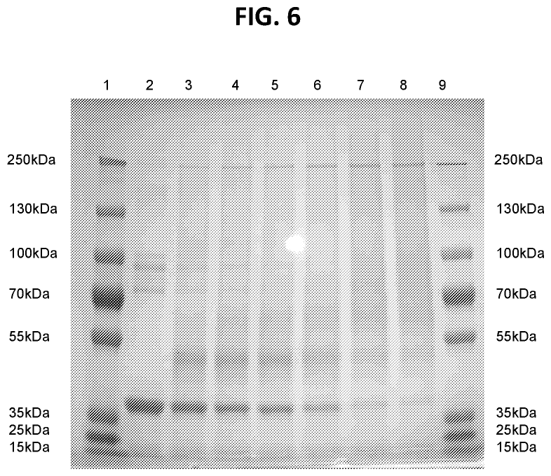

SDS-PAGE gel kinetics of Maleimide-PEG-SpA conjugate formation: SpA (1.0 mg) was suspended in PBS (1.0 mL at pH 7.4) and added directly to a microtube containing 1.0 mg or 2.0 mg of Maleimide-PEG₅₀₀₀-NHS, respectively. The mixture was vortexed for 20 seconds and put on an end-over-end rotator for mild rotation. The reaction was kept at room temperature for 1 hour. At 10, 30, and 60 minutes, aliquots (10 μL) were withdrawn, mixed with 40 μg of glycine (2 μL), and immediately added to 12 μL of 2× reducing Laemmli SDS gel loading buffer and placed directly into a water bath at 90° C. for 5 minutes to denature the protein. Control (time zero) samples were taken prior to mixing with the PEG. 10 μL of each sample was loaded on a 7.5% SDS-PAGE gel. Following electrophoresis, the gel was stained with PAGE-Blue protein stain, washed, and imaged (FIG. 6).

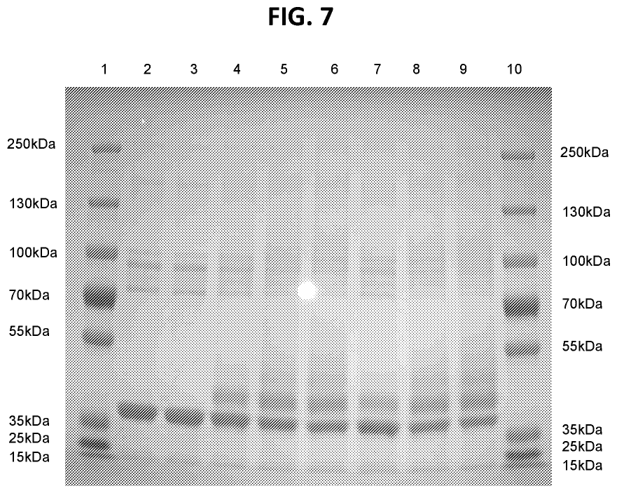

SDS-PAGE gel kinetics of Azido-PEG-SpA conjugate formation: SpA (1.0 mg) was suspended in PBS (1.0 mL at pH 7.4) and added directly to a microtube containing 0.4 mg or 0.8 mg of Azido-PEG₂₀₀₀-NHS, respectively. The mixture was vortexed for 20 seconds and put on an end-over-end rotator for mild rotation. The reaction was kept at room temperature for 1 hour. At 10, 30, and 60 minutes, aliquots (10 μL) were withdrawn, mixed with 40 μg of glycine (2 μL), immediately added to 12 μL of 2× reducing Laemmli SDS gel loading buffer, and placed directly into a water bath at 90° C. for 5 minutes to stop the reaction and denature the protein, respectively. Control (time zero) samples were taken prior to mixing with the bifunctional PEG. 10 μL of each sample was loaded on a 7.5% SDS-PAGE gel. Following electrophoresis, the gel was stained with PAGE-Blue protein stain, washed, and imaged (FIG. 7).

Quantification of free primary amines on human RBC surface: 10, 100, or 500 μg of sulfo-Cyanine5-NHS ester were added to 5×10⁶ of human RBCs suspended in PBS (pH 7.4) in microtubes to reach a total volume of 1.0 mL. The mixture was vortexed for 10 seconds and put on an end-over-end rotator for mild rotation. The reaction was kept at room temperature for 1 hour. The cells were then washed 4 times with PBS buffer and resuspended in PBS. The modified cells and blank cells were counted by a MOXI-Z cell counter (Orflo Technologies, Ketchum, Id.), and the fluorescence intensity was measured using a Tecan M1000 plate reader (Tecan Group Ltd., Switzerland) with excitation 646 nm and emission 664 nm. Serially diluted sulfo-Cyanine5 carboxylic acid in PBS was measured concurrently as the standard curve. The number of free primary amino groups on RBC cells was calculated based on the standard curve.

Quantification of free thiols on the surface of human RBCs: 50, 100, or 200 μg of Traut's reagent were added to 5×10⁶ of human RBCs suspended in PBS (pH 7.4) in microtubes to reach total a volume of 500 μL. The mixture was vortexed for 10 seconds and put on an end-over-end rotator for mild rotation. The reaction was kept at room temperature for 1 hour. The cells were then washed 3 times with PBS buffer and resuspended in 1 mL of PBS. 15 μg of sulfo-Cyanine5 maleimide or sulfo-Cyanine5 carboxylic acid were added into each sample. The reaction was allowed to continue for 2 hours under mild rotation at room temperature. Afterward, the cells were washed 4 times with PBS in the dark and resuspended in PBS. The modified cells and blank cells were counted by a cell counter and the fluorescence intensity was measured using a Tecan M1000 plate reader with excitation 646 nm and emission 664 nm. Serially diluted sulfo-Cyanine5-maleimide in PBS was measured concurrently as the standard curve. The number of free thiol groups on blank cells or Traut's reagent modified cells was calculated based on the standard curve.

Synthesis of RBC-PEG-SpA complexes—amino modification by N-hydrosuccinidyl ester-functionalized homobifunctional poly(ethylene glycol) (NHS-PEG-NHS): To synthesize RBC-PEG-SpA complexes through amino groups, SpA was dissolved in phosphate buffered saline (PBS, pH 7.4) and added to an 8.4-fold molar excess of dry NHS-PEG₅₀₀₀-NHS (typically 1.0 mg of SpA to 1.0 mg of PEG). The SpA/PEG solution was added to an equal volume of PBS containing varying numbers of RBCs within 1 minute. The reaction was mixed with an inversion mixer at 15 RPM for 10 minutes, and the cells were then washed 3 times and resuspended in PBS. (FIG. 2A).

Synthesis of RBC-PEG-SpA complexes—direct thiol modification by heterobifunctional Maleimide-PEG-NHS ester (MAL-PEG-NHS): To synthesize RBC-PEG-SpA complexes through reactions between maleimide and free thiol groups, NHS-PEG₅₀₀₀-maleimide was first reacted with SpA in PBS (pH 7.4) for 1 hour. Unreacted PEG was removed by diafiltration 3 times (MWCO 30k) to yield SpA-PEG-maleimide in PBS. The SpA-PEG-maleimide was added directly to RBCs and mixed with an inversion mixer for 2 hours. Cells were washed 3 times and resuspended in PBS. (FIG. 2B).

Synthesis of RBC-PEG-SpA complexes—converted thiol modification by heterobifunctional Maleimide-PEG-NHS ester (MAL-PEG-NHS): To synthesize RBC-PEG-SpA complexes through reactions between maleimide and converted thiol groups, NHS-PEG₅₀₀₀-maleimide was first reacted with SpA in PBS (pH 7.4) for 1 hour and the unreacted PEG was removed by diafiltration 3 times (MWCO 30k) to yield SpA-PEG-maleimide in PBS. Human RBCs were incubated with Traut's reagent at different concentrations on an inversion mixer at 15 RPM for 1 hour, and the cells were then washed 3 times and resuspended in PBS. SpA-PEG-maleimide was added to Traut's modified cells and mixed with an inversion mixer for 2 hours. Again, cells were washed 3 times and resuspended in PBS. (FIG. 2C).

Synthesis of RBC-PEG-SpA complexes—click chemistry modification by heterobifunctional Azido-PEG-NHS ester (N3-PEG-NHS) and Alkyne-PEG-NHS ester (Alk-PEG-NHS): To synthesize RBC-PEG-SpA conjugates through click reactions, azido-PEG₂₀₀₀-NHS was reacted with SpA in PBS (pH 7.4) for 1 hour and the unreacted PEG was then removed by diafiltration 3 times (MWCO 30k) to yield SpA-PEG-azido in PBS. Concurrently, human RBCs were reacted with alkyne-EPG₃₀₀₀-NHS with an inversion mixer at 15 RPM for 1 hour. Next, the cells were washed 3 times and resuspended in PBS. Then SpA-PEG-azido was added to RBC-PEG-alkyne, supplemented with CuSO₄/BTTAA and sodium ascorbate to initiate click reactions (Uttamapinant et al., supra). The reaction was mixed with an inversion mixer for 2 hours at room temperature. Finally, the cells were washed 3 times and resuspended in PBS. (FIG. 2D).

Binding of Antibody to RBC-PEG-SpA complexes: RBC-PEG-SpA complexes were suspended in 1 ml of PBS and the cell number was determined on a MOXI-Z cell counter (Orflo Technologies, Ketchum, Id.). An aliquot of the cells from the conjugation reaction was diluted to 1 ml in PBS and incubated with 2 μg of an ALEXA FLUOR® 488 labeled human Fc fragment for 1.5 hours with an inversion mixer at 15 RPM. The cells were pelleted, washed 3 times, resuspended in PBS, and recounted. Fluorescence was measured (490 nm excitation, 520 nm emission) on a Synergy H1 Hybrid plate reader (BioTek, Winooski, Vt.). Opaque, black, 96 well plates (Costar) were loaded with 100 μL of samples containing 1-5×10⁴ cells and standards of known concentrations of ALEXA FLUOR® 488-Fc. The binding capacities of the cells were calculated as Fc molecules/cell (TABLES 1 and 2). Cells not used for Fc binding were incubated with 4 μg of anti-TNFα antibody, as described above, and used for TNFα scavenging. The viability of RBC-PEG-SpA-antibody was tested using a CELLTITER-GLO™ Luminescent cell viability assay kit (Promega, Madison, Wis.).

TNFα Scavenging: Known numbers (typically 1-5×10⁶) of modified RBCs (RBC-PEG-SpA-antibody) were suspended in 100 μL of PBS and mixed with another 100 μL of PBS containing 500 pg of TNFα in centrifuge tubes. The reaction tubes were rotated for 5 hours, at which time the cells were pelleted and the supernatants recovered. The concentration of free TNFα remaining in buffer was measured by ELISA (Invitrogen). By subtracting the amount of unbound TNFα remaining in buffer from 500 pg, the amount of TNFα bound to RBC-PEG-SpA-anti-TNFα conjugates was calculated (TABLES 1 and 2).

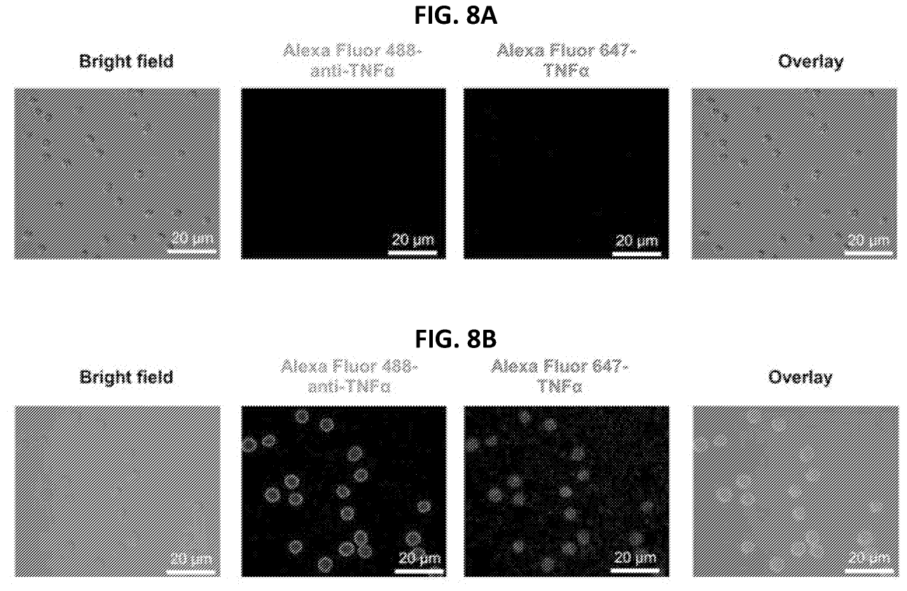

Confocal Microscopy: Modified RBCs and control cells were dispersed in 4-well LabTek II glass chamber slides (Fisher Scientific, Pittsburgh, Pa.) at a density of 100,000 cells per well in 0.1 mL of PBS. Images of RBCs from different storage times were captured with a Zeiss LSM 880 Meta FCS confocal microscope supplied with a 63×/1.40 NA oil-immersion lens (Dublin, Calif.). Single-fluorophore positive and cell-only negative controls were used to adjust the fluorescence intensity. The ALEXA FLUOR® 488 label was excited at 488 nm and emission was detected at 525 nm. The Cy5 and Alexafluor647 labels were excited at 643 nm and emission was detected at 670 nm. A minimum of three fields of view were used to collect images for each sample (FIGS. 8A, 8B, 9A-9C, and 10A-10H).

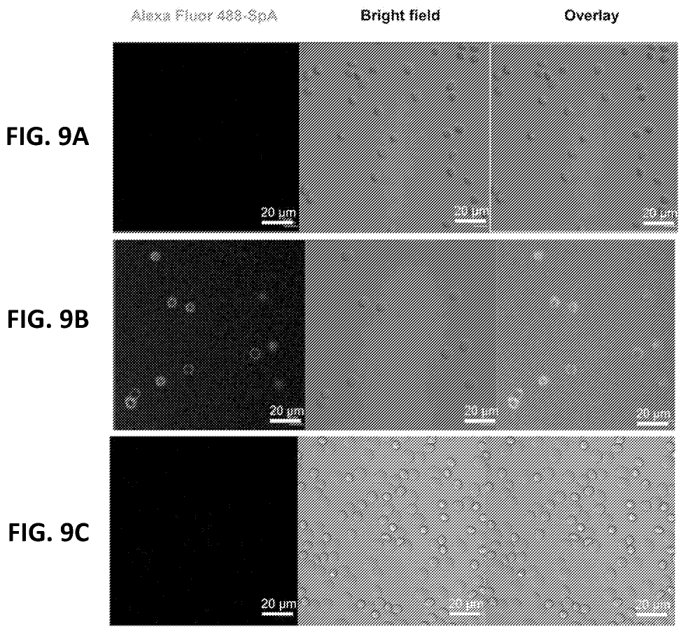

Fc exposure of RBC-PEG-SpA-antibody conjugates: SpA protein was labeled by ALEXA FLUOR® 488 using an ALEXA FLUOR® 488 Protein Labeling Kit (Fisher Scientific). RBC-PEG-SpA-rabbit antihuman TNF-α antibody conjugates were prepared as described above with a high density of IgG arrays (direct amine method). The cells were then washed three times with PBS. After washing, RBCs were incubated with 4 μg of ALEXA FLUOR® 488-SpA for 1.5 hours under rotation at room temperature. The cells were washed again with PBS before confocal imaging. Blank RBCs directly incubated with ALEXA FLUOR® 488-SpA were used as a control. RBC-PEG-rabbit antihuman TNF-α antibody conjugates without the involvement of SpA were also prepared using the direct amine method. These cells also were incubated with ALEXA FLUOR® 488-SpA and imaged after washing with PBS (FIGS. 9A-9C).

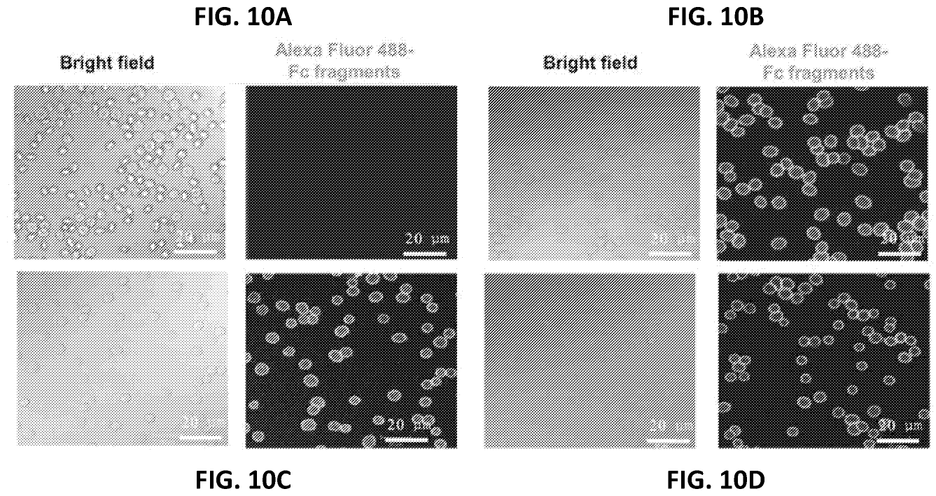

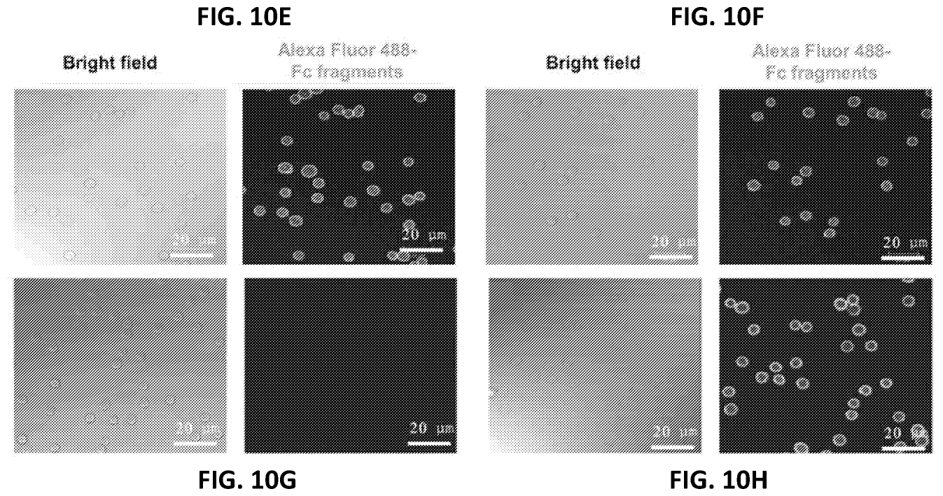

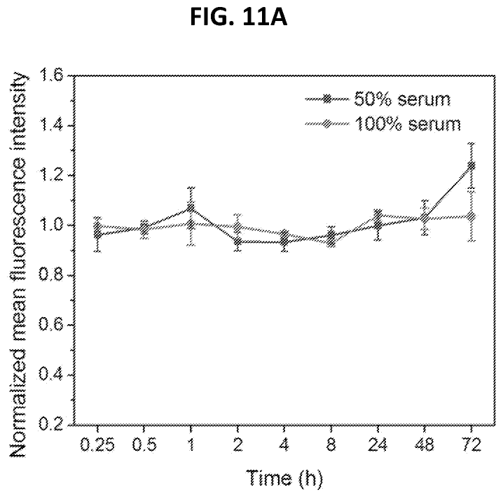

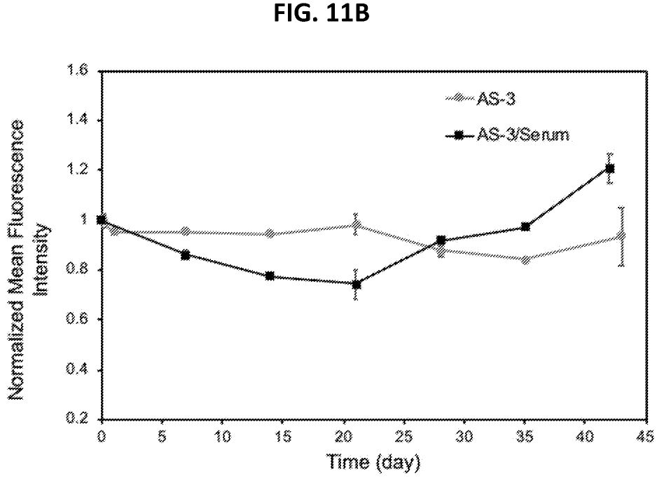

Stability of RBC-PEG-SpA-Antibody construct in vitro: RBC-PEG-SpA-IgG/ALEXA FLUOR® 488 constructs were made as described above, and the cells were then dispersed in PBS buffer containing 50% human serum or whole serum supplemented with 1% Pen-Strep. The cells were placed on an inversion mixer and rotated at 15 RPM under room temperature. At predetermined time points, aliquots were withdrawn, washed with FACS buffer (PBS containing 1% bovine serum albumin), and analyzed by a BD Accuri flow cytometer (BD Biosciences, San Jose, Calif.) (FIG. 11A). The percentage of ALEXA FLUOR® 488 positive cells was determined using Flowjo. The ALEXA FLUOR® 488 positive gate was drawn based on the blank RBC control, where the false positive frequency was restricted to below 0.1%. Confocal images also were collected at selected time points (FIGS. 10A-10H).

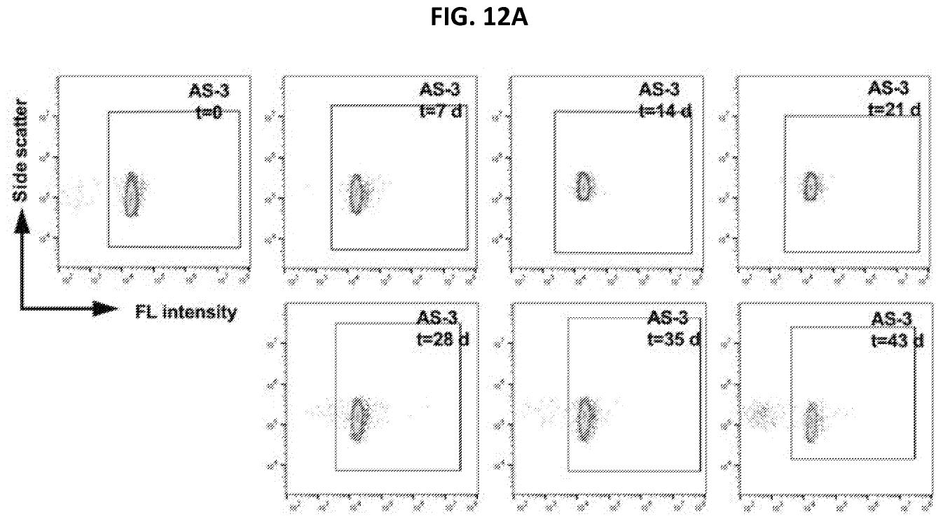

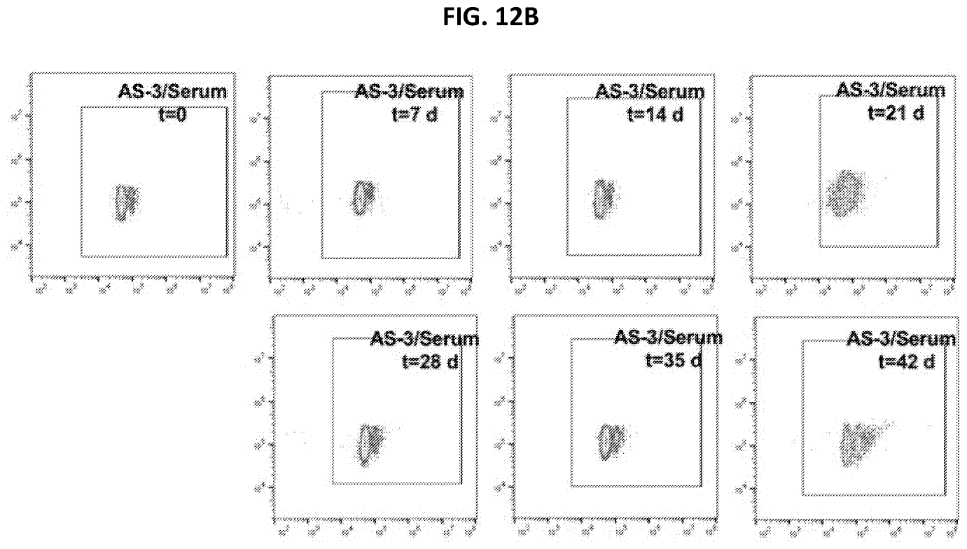

To investigate the long-term stability of RBC-PEG-SpA-IgG, the RBC-PEG-SpA-Fc/ALEXA FLUOR® 488 constructs were qualitatively examined in PBS buffer stored in a refrigerator by confocal microscopy at different time intervals. Freshly prepared RBC-PEG-SpA-IgG/ALEXA FLUOR® 488 constructs were then suspended in AS-3 buffer or AS-3 buffer containing 50% human serum, and the cells were stored in a refrigerator. At predetermined time points, aliquots were withdrawn, washed with FACS buffer, and analyzed by a BD Accuri flow cytometer as described above (FIGS. 11B, 12A, and 12B). Confocal images also were collected at selected time points.

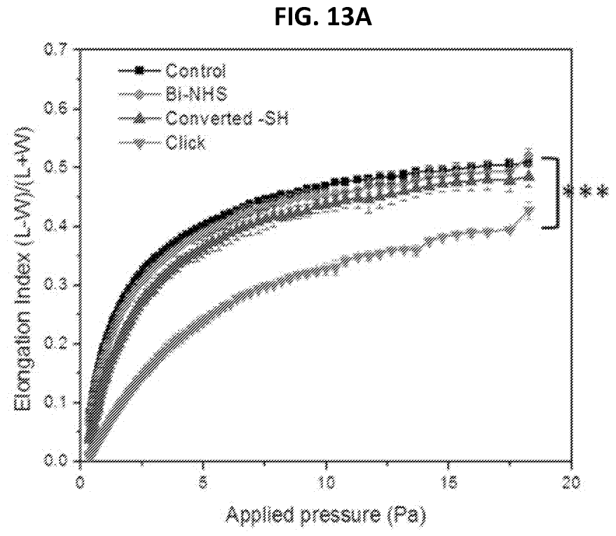

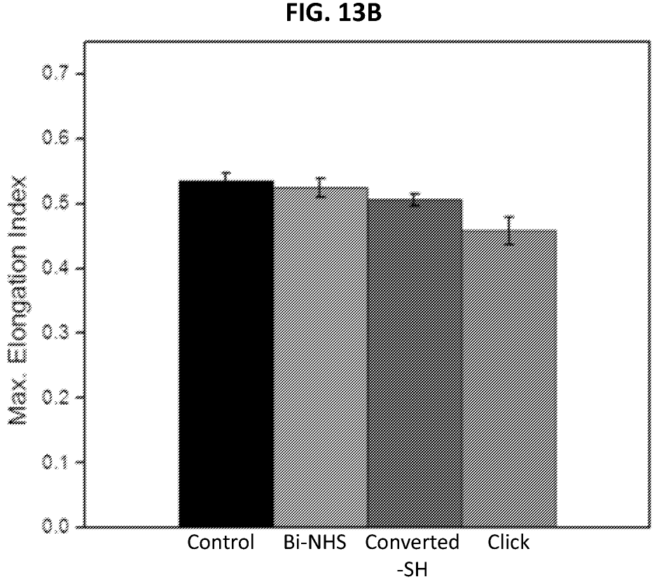

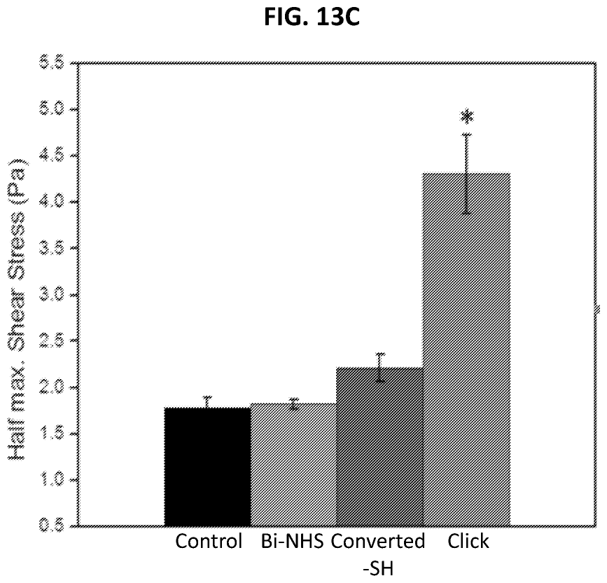

Deformability test of RBC-PEG-SpA-Antibody constructs: The deformability of RBCs and RBC-PEG-SpA-IgG were measured at room temperature using an ektacytometer (Rheo Meditech, South Korea) as described elsewhere (Pan et al., Sci Rep 8:1615, 2018). Briefly, 25 μL of 10% hematocrit suspension of RBCs or RBC-PEG-SpA-IgG was mixed with 675 μL of 5.5% (w/v) 360 kDa polyvinylpyrrolidone (Sigma Aldrich). Each RBC suspension was deposited into a flow channel and exposed to shear stress varying from 0 Pa to 18 Pa. The RBC suspensions under stress generated ellipsoidal diffraction patterns, which were recorded. The relationship between the shear stress applied and elongation of the diffraction index was analyzed. This analysis was used to determine the modeled maximum elongation index (EIₘₐₓ) and half maximum shear stress (SS_{1/2}) (FIGS. 13A-13C). Each experiment was repeated at least two times.

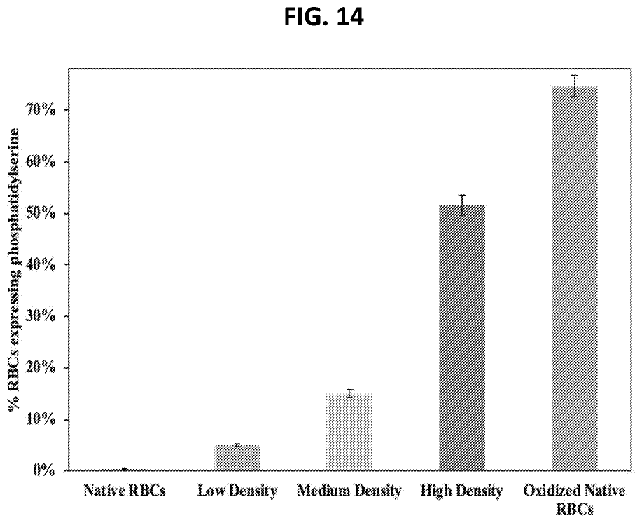

Effects of converted thiol modification on RBC exposure of phosphatidylserine: Membrane destabilization of RBC-PEG-SpA-IgG constructs through the converted thiol approach was assessed by monitoring phosphatidylserine exposure on RBCs using a commercially available Dead Cell Apoptosis Kit (Invitrogen). Low, medium, and high-density RBC-PEG-SpA-IgG constructs were briefly incubated at room temperature with phosphatidylserine-binding Annexin V-ALEXA FLUOR® 488 in a 2 mM CaCl₂) binding buffer for 15 minutes. Immediately after incubation, the fluorescence intensity of annexin V was measured using a BD Accuri flow cytometer (BD Biosciences, San Jose, Calif., USA). Native RBCs incubated in 0.2 mM CuSO₄/2.5 mM L-ascorbic acid for one hour were used as a positive control for induction of phosphatidylserine. Results were represented as a percentage of RBCs testing positive for phosphatidylserine exposure. Each experiment was carried out three times, and statistical analysis was performed (FIG. 14).

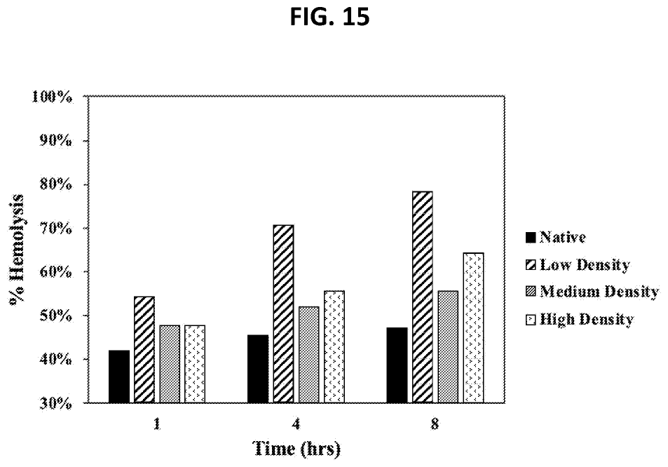

Effects of converted thiol modification on RBC hemolysis: Membrane destabilization of RBC-PEG-SpA-IgG constructs through the converted thiol approach was assessed by monitoring RBC sensitivity to mechanical stress. Equal numbers of the low, medium, and high-density RBC-PEG-SpA-IgG constructs were suspended in 1 mL PBS and were rotated at 24 RPM for 8 hours on an end-over-end rotator. At different time points, samples were centrifuged for 4 minutes at 13,400 g and supernatants were collected. Immediately following centrifugation, the release of hemoglobin from RBC hemolysis was measured by recording the absorbance of the supernatants at 540 nm with a UV-VIS spectrophotometer (Lambda 45, PerkinElmer). RBC hemolysis from suspension in 5 mM sodium phosphate buffer, pH8 was recorded as 100% RBC hemolysis (FIG. 15).

Statistical Analysis: Results are expressed as mean±SD unless otherwise noted. Significant differences between means were determined by two-tailed t-tests. p<0.05 was considered statistically significant.

### Example 2—Synthesis of Membrane Engineered RBC-PEG-SpA Conjugates

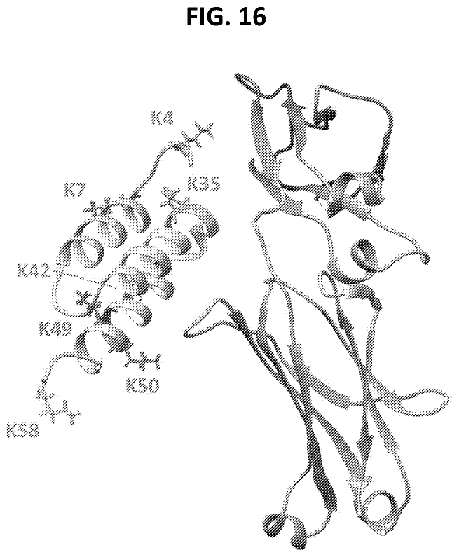

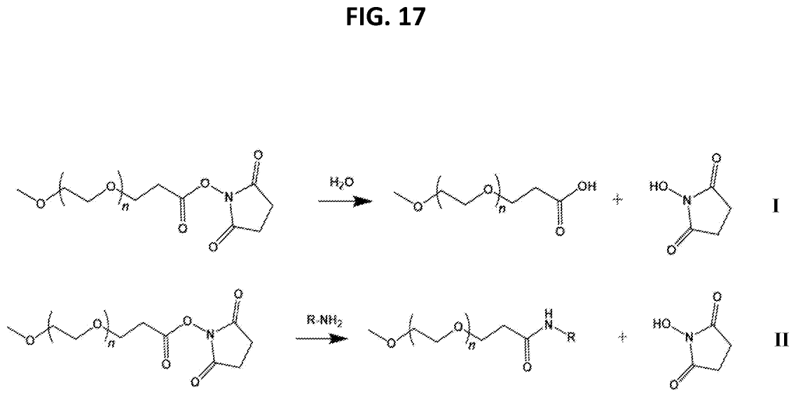

Site of attachment of PEG to SpA: Atom-transfer radical polymerization- (ATRP-) synthesized polymers can react with RBC membrane proteins to form stable cell-polymer conjugates (Clafshenkel et al., supra). To generate SpA-mediated anti-TNFα arrays on RBCs and identify the chemistry that produced the most stable and densely-modified constructs, four different attachment chemistries were designed. Each synthetic design began with the reaction of NHS-PEG-X, where X was either NHS, MAL, or azido, with accessible primary amines on the surface of SpA (part (i) of FIGS. 2A-2D). SpA (a 42 kDa protein) contains five homologous immunoglobulin binding domains (A-B-C-D-E), each of which can interact with the Fc region of antibodies (Graille et al., supra). It was important to understand which sites on SpA were likely being modified during the reaction with NHS-PEG-X, to ensure that the binding interface between SpA and antibody would not be disturbed. Studies were first conducted to determine how many sites in each domain are available for modification using NHS chemistry. The number of accessible primary amines on SpA was estimated using a small molecule model, NHS-Br (Murata et al., supra). NHS-Br reacted with the primary amines on SpA and the number of modifications was determined by MALDI-ToF MS by comparing the m/z value of native SpA with modified SpA. (FIG. 4). There were 35 accessible amino groups available for small molecule attachment. A tertiary-structure based prediction (Carmali et al., supra) was then performed on the crystal structure of the C domain of SpA in complex with the Fc fragment of human IgG (PDB: 4WWI) in order to determine which lysine amino groups were likely to be fast reacting sites (FIG. 16). Of the seven total lysines, one was predicted to be fast reacting (K58), four were predicted to be slow reacting (K4, K7, K35, K42), and two were predicted to be non-reacting (K49, K50). Moreover, none of the lysines were located at the interface where SpA-antibody binding occurs, suggesting that the NHS-PEG-X chemistry would not impede antibody binding. The NHS-PEG-X molecules that were used in the conjugation were much larger in size than the NHS-Br molecule that was used to model the reaction, so the number of achievable modifications with PEG was expected to be much reduced due to steric hindrance (Damodaran and Fee, Euro Pharm Rev 15:18-26, 2010). Analysis of the products from the SpA reactions with NHS-PEG-X by SDS-PAGE gels at increasing time intervals showed that each of the chemistries produced a modified protein product with mostly one PEG chain per protein and, to a lesser degree, two PEG chains per protein (FIGS. 5A, 5B, 6, and 7). From the tertiary structure-based prediction, it was most likely that K58 would be modified first, followed by one of the other fast-reacting lysines. From the SDS-PAGE gel, it also was evident that the reactions were completed within 10 minutes. This quick reaction time was verified through NHS hydrolysis and aminolysis spectroscopic studies of NHS-mPEG₅₀₀₀ (FIGS. 3 and 17). The hydrolysis reaction half-life of NHS-mPEG₅₀₀₀ was about 4 minutes, while the aminolysis reaction occurred within seconds. Thus, NHS-PEG-X only needed a few minutes of reaction time with SpA before the second conjugation reaction to RBCs could be performed to generate the final product.

Reaction chemistry: Initial studies were performed by first reacting NHS-PEG-NHS with SpA for a short time before exposing the reaction mixture to RBCs (FIG. 2A). By carefully controlling reaction time and stoichiometry, SpA was reproducibly coupled to RBC membrane proteins. Although these RBC-PEG-SpA conjugates were functional, the use of NHS-PEG-NHS would be expected to generate a mix of products that were impossible to analyze (protein multimers, aggregated RBCs and RBC-PEG). For this reason, a synthetic technique was developed that allowed separation of the PEG-SpA reaction from the reaction to couple that complex to RBCs (FIG. 2B).

In this synthetic technique, SpA was first reacted with NHS-PEG-MAL for 1 hour to generate a SpA-PEG-MAL protein-polymer conjugate. This reaction was performed at physiological pH (7.4) in PBS, such that the NHS-PEG-MAL would preferentially react with the amino groups on SpA via the NHS functional end (McCombs and Owen, supra; and Broyer et al., supra), leaving the MAL functional end free to react with thiol groups on the RBC surface during the second reaction. To optimize the reaction stoichiometry, NHS-PEG-MAL was reacted at different molar ratios with SpA. At a PEG:SpA ratio of 8:1, most of the SpA was conjugated to one or two PEG chains (as determined by an SDS-PAGE gel analysis) (FIG. 6). When the molar ratio was increased to 16:1, there were larger conjugates that possibly contained 3 to 4 PEG chains per SpA (FIG. 6, lanes 6-8). The higher grafting density of PEG chains would increase the likelihood of blocking the Fc binding domain of SpA, and as such, the lower PEG:SpA ratio of 8:1 was adopted reaction ratio for subsequent reaction with cells. After synthesis, the protein-polymer conjugates were purified from unreacted, and most likely hydrolyzed, excess NHS-PEG-MAL via diafiltration. SpA-PEG-MAL would then be able to directly modify free thiols on the RBC surface.

The numbers of free thiols and primary amines were determined next by fluorescence intensity measurements from reactions with sulfo-Cyanine5-MAL and sulfo-Cyanin2-NHS, respectively. The number of accessible free thiols on the surfaces of RBCs (3.6×10⁵/cell) was an order of magnitude lower than the number of accessible primary amines (5.4×10⁶/cell). When directly modifying the free thiols, therefore, the maximum grafting density would be significantly lower than what could be achieved by amine modification. To achieve both well-controlled chemistry and high grafting densities, a third approach to synthesizing RBC-PEG-SpA complexes was developed. In this approach, primary amines on the RBC surface were first converted to free thiols using Traut's reagent (FIG. 2C). The number of converted thiols was easily tuned by changing the reaction stoichiometry between Traut's reagent and primary amines. The number of thiol groups increased to 1.3×10⁶ (low Traut's reagent), 1.9×10⁶ (mid Traut's reagent), and 2.2×10⁶/cell (high Traut's reagent), and was thus on the same order of magnitude as the number of free primary amines on the RBC surface. After the amine conversion to thiols, remaining conjugation chemistries would be the same as the second approach.

Each of the synthetic approaches described above coupled a PEG-SpA complex directly to RBC membrane proteins. Additional studies were conducted to determine whether PEGylated RBCs could be created and then coupled to PEGylated SpA. Both components were PEGylated with PEG's that were terminated with the molecular participants in copper-catalyzed click chemistry (Besanceney-Webler et al., supra) (FIG. 2D). SpA primary amines were modified using an NHS-PEG-azido reagent. After 1 hour, the excess unreacted, hydrolyzed NHS-PEG-azido was removed via diafiltration. SDS-PAGE gel analysis showed that one to two PEG chains were conjugated to SpA. (FIG. 7). Concurrently, alkyne groups were incorporated onto the RBC membrane by reacting RBCs with an amine-specific NHS-PEG-alkyne. In this way, the SpA-PEG-azido could then be “clicked” to the RBC-PEG-alkyne to form the desired RBC-PEG-SpA complex.

Relationship between modification chemistry and degree of membrane modification: After synthesis of membrane engineered RBC-PEG-SpA constructs using the four approaches described above, the constructs were analyzed to determine how many molecules of antibody-accessible SpA were presented on the surface of each RBC. A fluorescently labeled Fc region of an IgG, ALEXA FLUOR® 488, was used to measure the binding capacity (TABLES 1 and 2). It was assumed that one molecule of SpA binds one molecule of IgG in these experiments. This assumption seemed reasonable given data from the use of immobilized SpA in antibody purification columns (Zhang et al., ACS Omega 2(4):1731-1737, 2017; and McCue et al., J Chromatogr A 989:139-153, 2003). For the NHS-PEG-NHS approach, the number of bound functional SpA molecules per RBC was controlled by changing reaction time and stoichiometry. The data displayed in TABLE 1 provide conditions that yielded between 1.1×10⁴ and 2.7×10⁵ functional SpA molecules per RBC. This surface coverage was equivalent to 1,930 SpA per square micron. The direct NHS-PEG-MAL chemistry, depicted in FIG. 2B, produced conjugates that could only bind 7.8×10⁴ SpA per RBC. This result was not surprising, as the presence of free thiols on RBC surfaces was known to be more than an order of magnitude less than the available primary amines. The converted NHS-PEG-MAL chemistry (FIG. 2C) produced conjugates that could bind 9.04×10⁵ SpA molecules per RBC when using the highest stoichiometry of Traut's reagent. Finally, the NHS-PEG-azido click chemistry approach produced conjugates that could bind 1.84×10⁵ SpA molecules per RBC, similar to the results of NHS-PEG-NHS. These numbers all agreed well with PEGylation densities reported elsewhere (Hashemi-Najafabadi et al., Bioconjug Chem 17:1288-1293, 2006).

### Example 3—Functionality of Membrane Engineered RBC-PEG-SpA Conjugates

Ultimately, the real tests of the RBC-PEG-SpA conjugates were to determine whether they could bind a target antibody, whether the RBC-displayed SpA-antibody complex could bind antigen, and whether the RBC-PEG-SpA-antibody complexes were stable. Using anti-TNFα as a model antibody, the theoretical antigen binding capacity of the RBC-PEG-SpA-anti-TNFα complexes was compared to the experimentally determined binding capacity as a function of the synthetic approach. The theoretical binding capacity in TABLES 1 and 2 assumed that each IgG bound two molecules of TNFα. Experimentally, TNFα was mixed with the RBC-PEG-SpA-antibody complexes, and after 5 hours, the cells were pelleted via centrifugation and the amount of TNFα remaining in the supernatant was determined with an ELISA assay. The bound TNFα was calculated by subtracting the amount of TNFα remaining in the supernatant from the initial amount of TNFα. It was discovered that about 25% of RBC-linked SpA generated using the NHS-PEG-NHS synthetic technique was able to bind to the antibody. The binding efficiency ([SpA_{bound}]/[SpAₜₒₜₐₗ]) using both thiol-directed synthetic strategies was about 10%, and the binding efficiency of the click chemistry was almost 20%. The membrane location of each point of SpA attachment was not controlled in the chemistry described herein. It was not surprising, therefore, that some fraction of the covalently coupled SpA would be located in a region that reduces the availability of antibody binding sites on SpA. These results confirmed that 10⁴-10⁵ functional antibody molecules/cell could be immobilized on RBC membranes. In every case, RBCs were generated that straightforwardly sequestered TNFα on RBC membranes without apparent hemolysis. Whole blood contains 5×10⁹ cells/mL, and thus, in just 10 mL of blood, about 1.4 mg of fully functional anti-TNFα was attached to RBCs. As a comparison, Humira is provided to patients at least biweekly in doses of 40 mg, with an elimination half-life of on the order of ten days. If the elimination half-life of RBC-PEG-SpA-anti-TNFαcomplexes matched those of native RBCs, it is reasonable to expect that a patient could be dosed with less antibody and/or at less frequent intervals. A key issue, then, was the stability and functionality of the RBC's once membrane-engineered, and the stability of the RBC-PEG-SpA conjugates and the RBC-PEG-SpA-anti-TNFα complexes.

### Example 4—Functionality of RBC-PEG-SpA-Anti-TNFα Complexes

Confocal imaging was used to directly observe the binding of the antigen to the antibody in RBC-PEG-SpA-anti-TNFα complexes. The RBC-PEG-SpA-anti-TNFα constructs synthesized using the NHS-PEG-NHS approach (high density) were fluorescently tagged with ALEXA FLUOR® 488 (green), and TNFα was fluorescently tagged with ALEXA FLUOR® 647 (red). The two components were allowed to associate for 2 hours while rotating. Samples were then pelleted and washed prior to confocal imaging (FIGS. 8A and 8B). The antibody signals were directly overlaid on the antigen signals on the RBC surfaces, evidencing strong association.

One might reasonably ask why an antibody could not be covalently attached to the surface of the RBC instead of on top of a SpA pillar. It was hypothesized that the potentially problematic impact of an exposed Fc region in antibody-antigen complexes could be minimized in a SpA-mediated attachment strategy. Indeed, the SpA-based strategy could mask the Fc region of attached antibodies. Therefore, RBC-PEG-SpA-antibody complexes were generated and exposed to fluorescently labeled SpA, which would only bind to exposed Fc domains. For RBC-PEG-SpA-anti-TNFα conjugates, no exposed Fc region was detected with a sample size of 5 million cells, each cell carrying about 100,000 modifiers (FIG. 9C). RBC-PEG-antibody complexes were then generated by replacing SpA with antibody in the membrane engineering experiment. As predicted, direct attachment of ALEXA FLUOR® 488 tagged anti-TNFα to RBCs did not damage the RBCs, but did result in exposure of Fc domains, as indicated by a strong green fluorescence signal (FIG. 9B). Thus, the SpA-mediated antibody arrays on RBCs combined the advantages of SpA as an independent therapeutic molecule (Bernton and Haughey, Basic Clin Pharmacol Toxicol 115:448-455, 2014) with its ability to lock antibodies on the surface of the RBC while masking the Fc region from the immune system during antigen binding.

### Example 5—In Vitro Stability of RBC-PEG-SpA-Anti-TNFα Complexes

Further studies were conducted to explore the key issue of storage stability of membrane engineered RBCs. RBC membrane proteins are not generally turned over by the cell since RBCs are enucleated. Thus, covalently bound SpA might be stably displayed on the membrane surface for times that exceeded the elimination half-life of soluble antibodies. The in vitro stability of the RBC-PEG-SpA-ALEXA FLUOR® 488-Fc complexes, synthesized using the NHS-PEG-NHS approach, was measured by fluorescent confocal microscopy at increasing time points, up to 65 days. Immediately after ALEXA FLUOR® 488-Fc binding, the complexes were observed under confocal microscopy (FIG. 10B). Constructs were stored in PBS buffer, pH 7.4, and imaged again on days 8, 14, 21, 44, and 65 (FIGS. 10C-10F and 10H), showing a strong fluorescent signal indicating that the antibody fragments remained associated with the RBC-PEG-SpA constructs. Moreover, the RBCs over the course of 65 days displayed similar morphologies to those on day 0. Unmodified RBCs on day 0 and day 65 were used as controls (FIGS. 10A and 10G). Thus, the stability of the RBC-PEG-SpA bond is clearly on the order of magnitude of cell lifetimes.

An issue to consider before large animal testing of in vivo stability was the stability of the SpA-antibody bond during storage in an immunoglobulin-free buffer and immunoglobulin-rich media. In circulation, the concentration of immunoglobulins exceeds 10 mg/ml, so studies were conducted to determine whether an RBC-displayed antibody would exchange with serum immunoglobulins, and if so at what timescale such an exchange would occur. Research during antibody purification optimization has shown that the binding of IgG to SpA is a reversible process, and that bound IgG can be replaced by competing antibodies based on their associated binding strengths (Weinberg et al., Biotechnol Bioeng 114:1803-1812, 2017). In human IgG the binding strength of antibodies to SpA follows the order IgG₁>IgG4>IgG2 (Choe et al., Materials (Basel) 9(12):994, 2016). Naturally, no one had studied whether RBC-bound antibodies, coupled to the membrane through an SpA linker, could be rapidly displaced by antibodies in plasma. To determine the rate of antibody exchange on the RBC constructs, fluorescently labeled RBC-PEG-SpA-ALEXA FLUOR® 488-Fc samples were synthesized via the converted thiol approach (FIG. 2B). While the NHS-PEG-NHS approach was used for the characterization studies described above, this approach is not well defined and could only be used to qualitatively track stability. The converted thiol approach provides greater control over modification density, and was thus the chosen construct for this experiment. The modified RBCs were incubated in buffers containing 0%, 50%, or whole serum (FIG. 11A) for 3 days. Samples also were incubated in 100% AS-3 solution or 50% AS-3 solution/50% whole serum supplemented with 1% Pen-Strep (FIG. 11B) for 43 days. It was important to determine antibody exchange rates in serum and the AS-3 storage buffer since they have substantially different pH's (pH 7.4 and 5.5, respectively) (D'Amici et al., Blood Transfus 10(Suppl 2):546-54, 2012). Fluorescence intensity at increasing time points was measured using flow cytometry with representative scatter plots (FIGS. 12A and 12B). The RBC-PEG-SpA-ALEXA FLUOR® 488-Fc constructs were stable in both 50% serum and whole serum over the 3-day study. In 100% AS-3, there were no competing antibodies in solution and the fluorescence signal remained constant over the 42 days, meaning that the Fc fragment remained associated with the construct. Dissociation of antibody from SpA can occur between pH 3-4 due to protonation of secondary amines at the binding domains that weaken the noncovalent interactions (Li et al., BMC Neurosci 8:22, 2007). It is noteworthy that, even under the slightly acidic conditions of the AS-3 solution (pH 5.5), the complexes remained associated with the originally bound antibodies for a long time. In 50% AS-3/50% whole serum, the constructs were also stable for approximately 42 days. Moreover, even in the presence of competing antibodies, the RBC-PEG-SpA-ALEXA FLUOR® 488-Fc construct remained intact. Interestingly, a second population of modified RBCs with a higher fluorescence intensity was observed in the scatter plots (FIG. 12B). It is not clear why there was a second group within the RBC population, but it should be noted that not all RBCs in a given population are at the same stage of their life span. It is possible that a certain percentage of RBCs presented more surface primary amines groups than others. After modification, this population of RBCs would be more densely modified with IgG, and thus a stronger fluorescence signal in the scatter plot would be observed. Regardless, the above results suggested that the majority of the RBC-PEG-SpA-antibody constructs remained stable and retained their ability to bind TNFα in vivo.

### Example 6—Structural Stability of RBC-PEG-SpA-Antibody Constructs

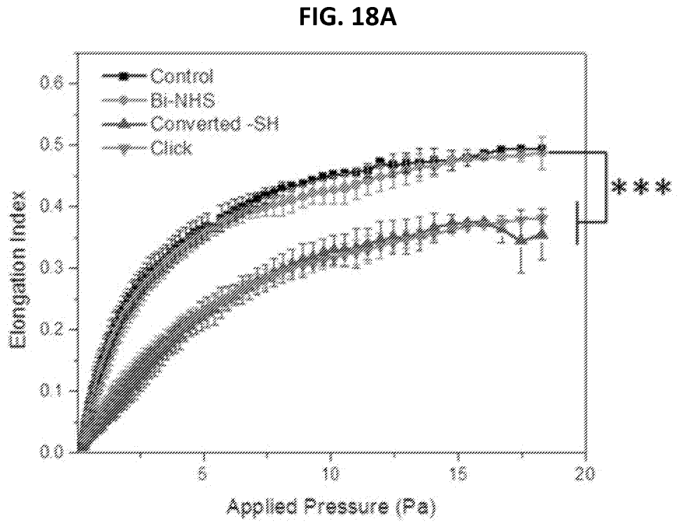

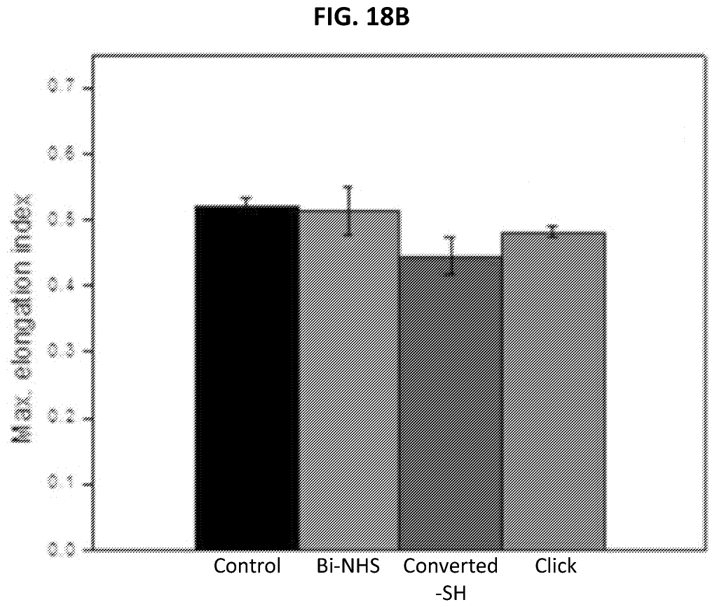

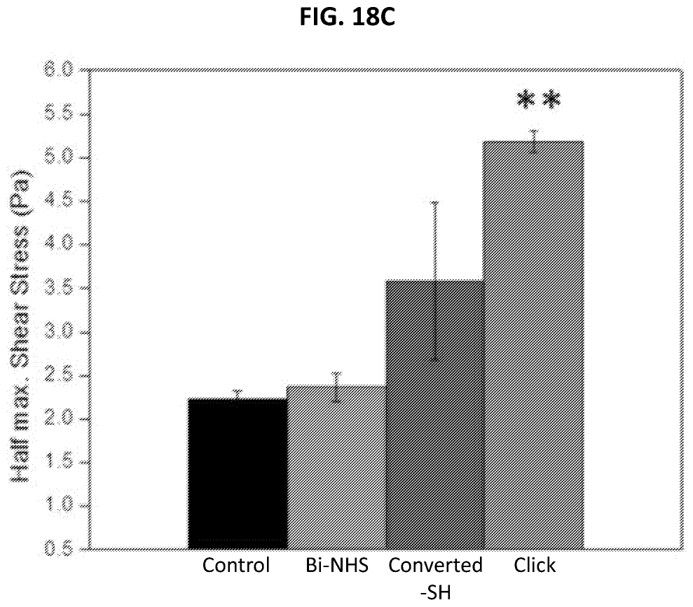

A benefit of the approach described herein for conjugating therapeutics to the RBC surface is a reduction in the negative effects on membrane stability that can occur during osmotic shock drug loading (Muzykantov, Expert Opin Drug Deliv 7:403-427, 2010). The mechanical properties of RBCs are directly linked to their function in circulation, because RBCs must be able to change their shape dramatically and reversibly as they experience different shear stresses while traveling through narrowed vasculature. The ability to change shape without hemolysis is due to the structural integrity of the RBC membrane (Jay, Biophys J 13:1166-1182, 1973; Lux, Blood 127:187-199, 2016; and velc and Svetina S, Cell Mol Bio Lett 17:217-227, 2012). To determine the effect of surface modification on RBC mechanical properties, the deformability of the RBC-PEG-SpA-antibody constructs, synthesized by three out of the four approaches, was measured using ektacytometry at low and high modification densities (number of IgG/cell×10⁴). Due to its lower modification density compared to the other three methods, the direct thiol approach was excluded from the experiment. In general, as the applied pressure increased, the RBCs were deformed and their elongation indexes increased accordingly (FIG. 13A). At low density, the RBC constructs synthesized via the NHS-PEG-NHS (Bi-NHS) approach (1.1±0.1 (×10⁴) IgG/cell) and via the converted thiol approach using NHS-PEG-MAL (1.0±0.2 (×10⁴) IgG/cell) were not significantly different from unmodified RBCs (control) in their mechanical response to an applied force (FIGS. 13A and 13B). The click chemistry approach (0.3±0.1 (×10⁴) IgG/cell), however, produced RBC constructs that were more rigid, and their ability to fully deform was diminished. The shear stress at half-max was also calculated from the elongation index versus applied pressure curves (FIG. 13C). It is evident from FIG. 13C that the click chemistry approach caused the mechanical properties of the RBCs to significantly change, possibly due to toxicity of the copper catalyst. It also is possible that the sodium and ascorbic acid used in the click chemistry method could have diffused into the RBCs and been oxidized by the ferric hemoglobin, which would cause the RBCs to lose their functionality of carrying oxygen. The other synthetic approaches did not significantly affect the RBC mechanical properties at low modification density. At high modification density, the Bi-NHS approach (2.3±0.3 (×10⁴) IgG/cell) was the only approach that did not cause decreased deformability in comparison to unmodified RBCs (FIGS. 18A-18C). The converted thiol approach (8.3±1.2 (×10⁴) IgG/cell) and the click reaction approach (2.4±0.1 (×10⁴) IgG/cell) had similar deformation profiles but were both less flexible than unmodified RBCs.

The RBC membrane is built from a basic triangular network of α- and β-spectrin molecules connected to band 3, ankyrin and protein 4.1 (Chasis et al., J Clin Invest 75:1919-1926, 1985). This network provides the RBC with stability, but also with the ability to deform. It is likely that when too many polymer chains are conjugated to these membrane proteins, their skeletal structure becomes partially rigid and they lose some mechanical flexibility. The hemolysis ratio and phosphatidylserine (PS) externalization of the thiol converted RBCs were also tracked to assess membrane destabilization. The data showed that this modification approach had minimal side effects on membrane integrity. The high-density modified cells showed the highest expression of phosphatidylserine externalization, while the low-density modified cells were more sensitive to mechanical stress (FIGS. 14 and 15). Overall, these results demonstrated that the RBCs remained intact upon modification and could deform naturally using appropriate modification density and chemistry. Since the RBC size is not generally being changed with this chemistry, the constructs would be expected to deform naturally when flowing through the vasculature in vivo.

### Example 7—Lifetime of Rat RBC-ALEXA FLUOR® 647

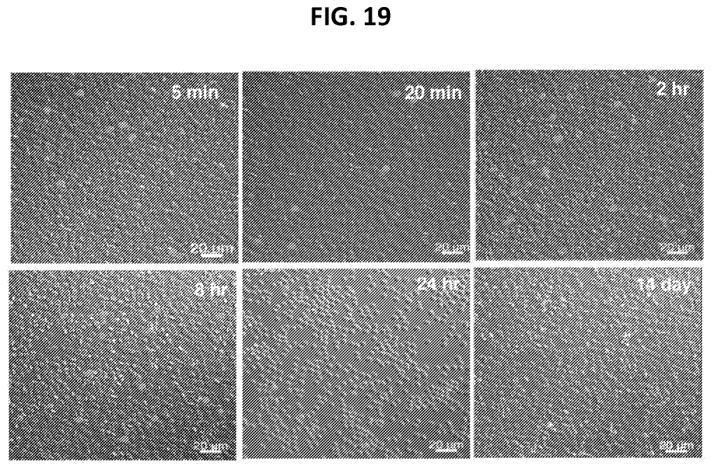

The exciting in vitro results implied that the RBC's with SpA-mediated antibody arrays had identical functional properties to naked RBCs. Rat RBCs have a lifetime of about 60 days (d'Almeida et al., supra). This translates to a half-life of about 14 days for blood drawn for transfusion, as processing and storage of blood increases the clearance rate, further reducing the observable half-life (d'Almeida et al., supra). Thus, it was hypothesized that the maximum observable lifetime of the RBC-PEG-SpA-antibody conjugates in a rat would be on the order of days. Two (2) ml of whole blood was extracted from a rat that had been fitted with an external jugular cannula. The RBCs were counted and modified with NHS ALEXA FLUOR® 647 label as described above. Next, 1 mL of the modified RBCs were injected back into the rat, and 0.3 mL blood samples were withdrawn over time. Direct confocal imaging was used to determine the fate of the injected cells. As expected, control unmodified RBCs showed no signal. In contrast, confocal microscopy clearly showed that the labelled RBC-ALEXA FLUOR® 647 were well tolerated in the rat over at least one day (FIG. 19).

Thus, the above studies included the design, synthesis, and testing of RBC-based platforms for carrying antibody-based therapeutic drugs using four different synthetic approaches that targeted either amines or thiols on the RBC surface. About 100,000 sites on the surface of each RBC were modified with antibodies. The high modification density, tunability, controlled synthesis, and resulting stability using the defined converted thiol approach was particularly promising, making it a strong candidate for future in vivo examination.

The synthetic approach, which used SpA covalently tethered to the cell surface through a short-chained PEG linker, yielded RBC-bound antibodies in an orientation that presented the antigen binding site to the solution while hiding the Fc region from immune cell receptors and immune complex-induced complement fixation. Antibodies bound in this fashion are less likely to induce the formation of anti-drug antibodies, and less likely to be inhibited by already existing anti-drug antibodies in vivo. The RBC-PEG-SpA-antibody constructs were shown to scavenge antigen, TNFα, from solution. Further, the constructs were stable in PBS for up to 65 days, in acidic AS3-solution for over 43 days, and in serum with competitive antibody binding for over 42 days without detection of antibody dissociation. Finally, the RBC constructs displayed similar mechanical properties to unmodified RBCs, except for the construct synthesized via click chemistry. Given that all of the individual components the RBC-PEG-SpA-antibody conjugates are currently being tested or used clinically (Bourgeaux et al., Drug Des Devel Ther 10:665-676, 2016; Chang and Girgis, Aust Fam Physician 36:1035-1038, 2007; D'souza et al., Expert Opin Drug Deliv 13:1257-1275, 2016; and Messerschmidt et al., J Biol Response Mod 3:325-329, 1984), there is a likely path to clinical use of these conjugates. In vivo studies conducted in rodents have shown that rat RBCs also can be successfully modified while maintaining the ability to scavenge TNFα (TABLE 3).

TABLE 1
Amount of SpA molecules per RBC that can bind anti-TNFα and TNFα.

| | | Number of | Predicted TNFα | Measured TNFα bound by | |
|---|---|---|---|---|---|
| | | accessible SpA | binding capacity of | RBC-PEG-SpA-anti-TNFα | |
| | | molecules per RBC | 1 × 10⁶ cells | conjugates on 1 × 10⁶ RBCs | % predicted |
| Sample | Method | (×10⁵)^{a} | (ng monomeric TNFα)^{b} | (pg TNFα)^{c} | capacity^{d} |
| High density | NHS-PEG-NHS | 2.69 ± 0.15 | 15.19 ± 0.86 | 4232.68 ± 32.18 | 27.85 ± 0.02 |
| Mid density | NHS-PEG-NHS | 0.22 ± 0.03 | 1.24 ± 0.18 | 33.31 ± 0.86 | 2.69 ± 0.07 |
| Low density | NHS-PEG-NHS | 0.11 ± 0.01 | 0.63 ± 0.04 | 28.03 ± 1.96 | 4.45 ± 0.31 |
| High density | Direct: NHS-PEG-MAL | 0.78 ± 0.18 | 4.42 ± 1.02 | 449.18 ± 23.40 | 10.16 ± 0.53 |
| High density | Converted: NHS-PEG-MAL | 9.04 ± 1.32 | 51.08 ± 7.46 | 6306.83 ± 33.82 | 12.35 ± 0.07 |
| High density | NHS-PEG-azido | 1.84 ± 0.01 | 10.38 ± 0.06 | 1833.84 ± 13.17 | 17.66 ± 0.13 |

^{a}The modification density determined from measurements of the Fc-488 binding was reported as SpA molecules/cell and was as expected for the number of cells modified.
^{b}Potential TNFα binding capacity was calculated assuming one antibody per SpA and two monomeric TNFα molecules/antibody.
^{c}Samples were gently mixed with TNFα proteins for 5 hours and centrifuged to remove cells. Supernatants were analyzed by ELISA and reported as nanograms monomeric TNFα bound per 10⁶ cells. Control samples were TNFα alone and TNFα with unmodified cells.
^{d}% predicted capacity was calculated as the ratio of measured TNFα binding to the theoretical maximum TNFα binding capacity.

TABLE 2
Percentage of SPA molecules per cell that create TNF-α binding conjugates in screening conditions

| | | | | | Measured TNF-α bound | |
|---|---|---|---|---|---|---|
| | | | Number of | Predicted TNF-α | by RBC-PEG-SpA-anti- | |
| | | | accessible SpA | binding capacity of | TNFα conjugates on | |
| | | | molecules per cell | 1 × 10⁶ cells | 1 × 10⁶ RBCs | % Predicted |
| Sample | Species | Method | (×10⁵)^{a} | (ng monomeric TNFα)^{b} | (pg TNFα)^{c} | capacity^{d} |
| High density | human | Low Traut's | 2.46 ± 0.49 | 13.88 ± 2.78 | 2962.83 ± 15.01 | 21.35 ± 0.11 |
| High density | human | Mid Traut's | 5.72 ± 2.08 | 32.28 ± 11.74 | 4764.33 ± 90.17 | 14.74 ± 0.28 |
| High density | human | Low click | 1.10 ± 0.09 | 6.21 ± 0.51 | 821.76 ± 5.88 | 13.23 ± 0.09 |
| High density | human | Mid Click | 1.62 ± 0.04 | 9.16 ± 0.24 | 945.49 ± 4.30 | 10.32 ± 0.05 |

^{a}The modification density determined from measurements of the Fc-488 binding was reported as SpA molecules/cell and was as expected for the number of cells modified.
^{b}Potential TNFα binding capacity was calculated assuming one antibody per SpA and two monomeric TNFα molecules/antibody.
^{c}Samples were gently mixed with TNFα proteins for 5 hours and centrifuged to remove cells. Supernatants were analyzed by ELISA and reported as nanograms monomeric TNFα bound per 10⁶ cells. Control samples were TNFα alone and TNFα with unmodified cells.
^{d}% predicted capacity was calculated as the ratio of measured TNFα binding to the theoretical maximum TNFα binding capacity.

TABLE 3
Amount of SpA molecules per rat RBC that can bind anti-TNFα and TNFα.

| | | | | | Measured TNFα bound | |
|---|---|---|---|---|---|---|
| | | | Number of | Predicted TNFα | by RBC-PEG-SpA-anti- | |
| | | | accessible SpA | binding capacity of | TNFα conjugates on | |
| | | | molecules per cell | 1 × 10⁶ cells | 1 × 10⁶ RBCs | % predicted |
| Sample | Species | Method | (×10⁵)^{a} | (ng monomeric TNFα)^{b} | (pg TNFα)^{c} | capacity^{d} |
| High density | rat | NHS-PEG-NHS | 3.23 ± 0.27 | 18.27 ± 1.54 | 999.06 ± 13.28 | 5.47 ± 0.07 |
| Mid density | rat | NHS-PEG-NHS | 0.34 ± 0.17 | 1.91 ± 0.95 | 20.51 ± 3.92 | 1.07 ± 0.20 |
| Low density | rat | NHS-PEG-NHS | 0.12 ± 0.04 | 0.68 ± 0.24 | 20.30 ± 1.50 | 2.98 ± 0.22 |

^{a}The modification density determined from measurements of the Fc-488 binding was reported as SpA molecules/cell and was as expected for the number of cells modified.
^{b}Potential TNFα binding capacity was calculated assuming one antibody per SpA and two monomeric TNFα molecules/antibody.
^{c}Samples were gently mixed with TNFα proteins for 5 hours and centrifuged to remove cells. Supernatants were analyzed by ELISA and reported as nanograms monomeric TNFα bound per 10⁶ cells. Control samples were TNFα alone and TNFα with unmodified cells.
^{d}% predicted capacity was calculated as the ratio of measured TNFα binding to the theoretical maximum TNFα binding capacity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A conjugate comprising:
a red blood cell (RBC);
a synthetic polymer comprising at least first and second coupling moieties, wherein the polymer is coupled to a thiol group on the RBC via the first moiety; and
a biomacromolecule coupled to the polymer via the second moiety.

2. The conjugate of claim 1, wherein the polymer is coupled to an amino group on the RBC.

3. The conjugate of claim 1, wherein the polymer is, or is derived from:
(i)N-hydrosuccinidyl ester-functionalized homobifunctional poly(ethylene glycol) (NHS-PEG-NETS), or
(ii) azido-PEG-NHS ester (N3-PEG-NHS) and alkyne-PEG-NHS ester (Alk-PEG-NHS).

4. (canceled)

5. The conjugate of claim 1, wherein the polymer is, or is derived from:
(i) maleimide-PEG-NHS ester (MAL-PEG-NHS), or
(ii) 2-iminothiolane hydrochloride (Traut's reagent) and MAL-PEG-NHS ester.

6. The conjugate of claim 1, wherein the biomacromolecule is a polypeptide.

7. The conjugate of claim 6, wherein the polypeptide is staphylococcal protein A (SpA).

8. The conjugate of claim 1, wherein the conjugate further comprises a therapeutic polypeptide bound to the polymer-coupled biomacromolecule.

9. The conjugate of claim 8, wherein the therapeutic polypeptide is an antibody, and wherein the polymer-coupled biomacromolecule binds to the antibody such that the Fc region of the antibody is masked.

10. The conjugate of claim 1, wherein the conjugate comprises polymer molecules coupled to the RBCs at a density of about 10⁴ to about 10⁸ polymer molecules per RBC.

11. A method for coupling a biomacromolecule to a RBC, the method comprising:
(a) conjugating a polymer to the biomacromolecule via a first moiety of the polymer, wherein the polymer is MAL-PEG-NHS and wherein the first moiety is NHS, to generate a biomacromolecule-polymer-MAL conjugate,
(b) coupling Traut's reagent to one or more amino groups on the RBC thus generating thiol groups on the RBC, and
(c) coupling the biomacromolecule-polymer-MAL conjugate to a thiol group on the RBC via the MAL moiety of the polymer.

12. The method of claim 11, wherein each of steps (a), (b), and (c) is carried out in a separate reaction.

13. The method of claim 11, wherein the biomacromolecule is a polypeptide.

14. The method of claim 13, wherein the polypeptide is SpA.

15. The method of claim 11, further comprising contacting the polymer-conjugated biomacromolecule with a therapeutic polypeptide.

16. The method of claim 15, wherein the therapeutic polypeptide is an antibody.

17. The method of claim 11, wherein the polymer is conjugated to the RBC at a density of about 10⁴ to about 10⁸ polymer molecules per RBC.

18-38. (canceled)

39. A method for modifying a RBC, comprising contacting the RBC with Traut's reagent such that one or more amino groups on the RBC are effectively converted to thiol groups.

40. A RBC conjugated to Traut's reagent, wherein the RBC conjugated to Traut's reagent was obtained according to the method of claim 39.

41-50. (canceled)
